SEQUENCE LISTING (34 sequences)

SEQ ID NO: 1 - protein, 34 aa, Artificial
  Generic P-Superfamily Conopeptides
XXCXXXXCXXXXXCXXXCXXCXXXXXCXXXXXXX

SEQ ID NO: 2 - protein, 28 aa, Conus ammiralis
  PEPTIDE (1)..(28): Xaa may be Glu or Gla
SCNNSCNXHSDCXSHCICTFSGCKIILI

SEQ ID NO: 3 - protein, 27 aa, Conus ammiralis
  PEPTIDE (1)..(27): Xaa may be Glu or Gla
SCNNSCNXHSDCXSHCICTFRGCGAVN

SEQ ID NO: 4 - protein, 28 aa, Conus caracteristicus
  PEPTIDE (1)..(28): Xaa at residues 9 and 26 may be Glu or Gla; Xaa at residue 14 may be Pro or Hyp
ASCGGTCTXSADCXSTCSTCLHAQCXST

SEQ ID NO: 5 - protein, 25 aa, Conus caracteristicus
  PEPTIDE (1)..(25): Xaa at residues 8 and 25 may be Glu or Gla; Xaa at residue 13 may be Pro or Hyp
SCGGTCTXSADCXSTCSTCLHAQCX

SEQ ID NO: 6 - protein, 27 aa, Conus consors
  PEPTIDE (1)..(27): Xaa at residue 11 and 24 may be Glu or Gla; Xaa at residue 13 may be Tyr, [125]I-Tyr, mono-iodo-Tyr, di-iodo-Tyr, O-sulpho-Tyr or O-phospho-Tyr
ACTGSCNSDSXCXNFCDCIGTRCXAQK

SEQ ID NO: 7 - protein, 27 aa, Conus gloriamaris
SCNNSCQSHSDCASHCICTFRGCGAVN

SEQ ID NO: 8 - protein, 32 aa, Conus imperialis
  PEPTIDE (1)..(32): Xaa at residues 12, 14 and 27may be Pro or Hyp; Xaa at residue 18 may be Tyr, [125]I-Tyr, mono-iodo-Tyr, di-iodo- Tyr, O-sulpho-Tyr or O-phospho-Tyr; Xaa at residues 22 and 31 may be Glu or Gla
NGCNGNTCSNSXCXNNCXCDTXDDCHXDRRXH

SEQ ID NO: 9 - protein, 27 aa, Conus pennaceus
  PEPTIDE (1)..(27): Xaa at residue 6 may be Pro or Hyp; Xaa at residues 17 and 20 may be Glu or Gla
LTCNDXCQMHSDCGICXCVXNKCIFFM

SEQ ID NO: 10 - protein, 27 aa, Conus textile
  PEPTIDE (1)..(27): Xaa may be Glu or Gla
GCNNSCQXHSDCXSHCICTFRGCGAVN

SEQ ID NO: 11 - protein, 27 aa, Conus textile
  PEPTIDE (1)..(27): Xaa may be Glu or Gla
GCNNSCQXHSDCXSHCICTSRGCGAVN

SEQ ID NO: 12 - DNA, 20 bp, amplification primer
  misc_feature (1)..(20): n is inosine
ccrttnacngcnccrcancc

SEQ ID NO: 13 - DNA, 20 bp, amplification primer
  misc_feature (1)..(20): n is inosine
tgrcanswrttrttrcancc

SEQ ID NO: 14 - DNA, 20 bp, amplification primer
  misc_feature (1)..(20): n is inosine
atrcartgnswytcrcartc

SEQ ID NO: 15 - protein, 5 aa, Conus textile
FLEEL

SEQ ID NO: 16 - protein, 27 aa, Conus textile
  PEPTIDE (1)..(27): Xaa is unknown
GCNNSCQXHSDCXSHCICTFRGCGAVN

SEQ ID NO: 17 - DNA, 461 bp, Conus ammiralis
  CDS (7)..(270)
gttaaaatgcatctgtcactggcacgctcagctgttttgatgttgcttctgctgtttgccttgggcaactttgttgtggtccagtcaggacagataacaagagatgtggacaatggacagctcacggacaaccgccgtaacctgcaatcgaagtggaagccagtgagtctcttcatgtcacgacggtcttgtaacaattcttgcaatgagcattccgattgcgaatcccattgtatttgcacgtttagcggatgcaaaattattttgatataaacggattgagtttgctcgtcaacaagatgtcgcactacagctcctctctacagtgtgtacatcgaccaaacgacgcatcttttatttctttgtctgttgtatttgttttcctgtgttcataacgtacagagccctttaattacctttactgctcttcacttaacctgataaccggaaggtccagtgct

SEQ ID NO: 18 - protein, 88 aa, Conus ammiralis
MHLSLARSAVLMLLLLFALGNFVVVQSGQITRDVDNGQLTDNRRNLQSKWKPVSLFMSRRSCNNSCNEHSDCESHCICTFSGCKIILI

SEQ ID NO: 19 - DNA, 459 bp, Conus ammiralis
  CDS (7)..(270)
gttaaaatgcatctgtcactggcacgcttagctgttttgatgttgcttctgctgtttgccttgggcaactttgttgtggtccagtcaggacagataacaagagatgtggacaatggacagctcacggacaaccgccgtaacctgcaatcgaagtggaagccagtgagtctcttcatgtcacgacggtcttgtaacaattcttgcaatgagcattccgattgcgaatcccattgtatttgcacgtttagaggatgcggagctgttaatggttgagtttgctcgtcaacatgatgtcgcactacacactacagctcctctctacagtgtgtacatcgaccaaacgacgcatcttttatttctttgtctgttgtgtttgttttcctgtgttcataacgtacagagccctttaattacttttactgctcttcacttaacctgataaccagaaggtccagtgct

SEQ ID NO: 20 - protein, 88 aa, Conus ammiralis
MHLSLARLAVLMLLLLFALGNFVVVQSGQITRDVDNGQLTDNRRNLQSKWKPVSLFMSRRSCNNSCNEHSDCESHCICTFRGCGAVNG

SEQ ID NO: 21 - DNA, 422 bp, Conus caracteristicus
  CDS (7)..(258)
gttacaatgcatctgtcactggcacgctcagctgtcttgatgttgcttctgctgtttgccttggacaacttcgttggggtccagccaggacagataacaagagatgtggacaaccgccgtaaccggcaatcgcgatggaagccaaggagtctcttcaagtcacttcataaacgagcatcgtgtggagggacttgcacggaaagtgccgattgcccttccacgtgtagtacttgcttacatgctcaatgcgagtcaacatgatgtcgcactacagctcttctctacagtgtgtacatcgaccgtacgacgcatcttttatttctttggctgtttcattcgttttcttgtgttcataacatgcggagcccttccgttacctctactgctctacacttaacctgataaccagaaaatccagtact

SEQ ID NO: 22 - protein, 84 aa, Conus caracteristicus
MHLSLARSAVLMLLLLFALDNFVGVQPGQITRDVDNRRNRQSRWKPRSLFKSLHKRASCGGTCTESADCPSTCSTCLHAQCEST

SEQ ID NO: 23 - DNA, 426 bp, Conus caracteristicus
  CDS (7)..(252)
gttacaatgcatctgtcactggcacgctcagctgttttgatgttgcttctgctgtttgccttggacaacttcgttggggtccaaccaggacagataactagagatgtggacaaccgccgtaacctgcaatcgcgatggaagccaaggagtctcttcaagtcacttcataaacgagcatcgtgtggagggacttgcacggaaagtgccgattgcccttccacgtgtagtacttgcttacatgctcaatgcgagtgaacatgatgtcgcactacagctcttctctacagtgtgtacatcgaccgaccgtacgacgcatcttttatttctttgtctgtttcattcgttttcttgagttcataacatgcggagcccttccgttacctctactgctctacacttaagctgataaccagaaaatccagtact

SEQ ID NO: 24 - protein, 82 aa, Conus caracteristicus
MHLSLARSAVLMLLLLFALDNFVGVQPGQITRDVDNRRNLQSRWKPRSLFKSLHKRASCGGTCTESADCPSTCSTCLHAQCE

SEQ ID NO: 25 - DNA, 428 bp, Conus consors
  CDS (1)..(216)
atgttgcttctgctgtttgccttgggcatcttcgttggggtccagccagaacagataacaagagatgtggacaagggatactccacggatgatggccatgacctgctatcgctgttgaagcaaatcagtctccgcgcgtgtacagggtcttgcaatagtgacagcgaatgctacaatttctgcgactgcattgggaccagatgtgaggcacaaaagtagacgtcagaagaaaaggtcccagtcgctcaaggcaagaactaaacgtagagagtttccccgtcaacatgatgtcgcactacaacgctattctactgcgtgtatatcgaccaaacgacgcatcttttatttctttgtctgtttgagttgttttcgtgtgttccatttccatgacctttactgcccaacacttatcctgataaccagaaggt

SEQ ID NO: 26 - protein, 72 aa, Conus consors
MLLLLFALGIFVGVQPEQITRDVDKGYSTDDGHDLLSLLKQISLRACTGSCNSDSECYNFCDCIGTRCEAQK

SEQ ID NO: 27 - DNA, 450 bp, Conus gloriamaris
  CDS (27)..(290)
cccagaaaggaaacacagcggttaaaatgcatctgtcactggcacgctcagctgttttgatgttgcttctgctgtttgccttgggcaactttgttgtggtccagtcaggactgataacaagagatgtggacaatggacagctcacggacaaccgccgtaacctgcaaacggagtggaacccattgagtctcttcatgtcacgacggtcttgtaacaattcttgccagagccattccgattgcgcatcccattgtatttgcacgtttagaggatgcggagctgtcaatggttgagtttgctcgtcaacatgatgtcgcactacacactacagctcctctctacagtgtgtacatcgaccaaacgacgcatcttttatttctttgtctgttgtatttgttttcctgtgttcataacgtacagagccctttaattacctttactgctcttcac

SEQ ID NO: 28 - protein, 88 aa, Conus gloriamaris
MHLSLARSAVLMLLLLFALGNFVVVQSGLITRDVDNGQLTDNRRNLQTEWNPLSLFMSRRSCNNSCQSHSDCASHCICTFRGCGAVNG

SEQ ID NO: 29 - DNA, 524 bp, Conus imperialis
  CDS (7)..(285)
gttaaaatgcatctgtcactggcaagctcagctgctttgatgttgcttctgctttttgccttgggcaacttcgttggggtccagccaggacaaataagagatctgaacaaaggacagctcaaggacaaccgccgtaacctgcaatcgcagaggaaacaaatgagtctcctcaagtcacttcatgatcgaaatgggtgtaacggcaacacgtgttccaatagcccctgccctaacaactgttattgcgatactgaggacgactgccaccctgacaggcgtgaacattagagattagagagtttccttgtcaacatgatgtcgcaccacacctctgctctgcagtgtgtacatcgaccagtcgacgcatctgttatttctttgtctgttggattgtacatcgaccagtccacgcatctgttatttctttgtctgtttgatttgttttcgtgtgttcataacacacagagcctttctattatctgtattgcaatacactttgcctgataaccagaaagtccagtgct

SEQ ID NO: 30 - protein, 93 aa, Conus imperialis
MHLSLASSAALMLLLLFALGNFVGVQPGQIRDLNKGQLKDNRRNLQSQRKQMSLLKSLHDRNGCNGNTCSNSPCPNNCYCDTEDDCHPDRREH

SEQ ID NO: 31 - DNA, 450 bp, Conus pennaceus
  CDS (1)..(234)
atgttgcttctgctgtttgccttgggcagcttcgttgtggtccagtcaggacagataacaagagatgtggacaatgggcagctcgcggacaaccgccgtaccctgcgatcgcagtggaagcaagtgagtttcttcaagtcacttgataaacgactgacttgtaacgatccttgccagatgcattccgattgcggcatatgtgaatgcgtggaaaataaatgcatatttttcatgtaaacggattgagtttgcttgtcaacacaatgtcgcactgcagctcttctctaccggtgggtacatcgaccaaacgacgcatcttttatttctttgtctgtttcgtttgttctcctgtgttcataacgtacagagccctttaactacccttactgctcttcacttaacctgataacctgaaggtccggtgcagctggcgtagccttcacagtttcg

SEQ ID NO: 32 - protein, 78 aa, Conus pennaceus
MLLLLFALGSFVVVQSGQITRDVDNGQLADNRRTLRSQWKQVSFFKSLDKRLTCNDPCQMHSDCGICECVENKCIFFM

SEQ ID NO: 33 - DNA, 811 bp, Conus textile
  CDS (28)..(294)
acccagaaaggaaacacagcggttaaaatgcatctgtcactggcacgctcagctgttttgatgttgcttctgctgtttgccttgggcaactttgttgtggtccagtcaggacagataacaagagatgtggacaatggacagctcacagacaaccgccgtaacctgcaatcgaagtggaagccagtgagtctctacatgtcacgacggggttgtaacaattcttgccaggagcattccgattgcgaatcccattgtatttgcacgtttagaggatgcggagctgttaatggttgagtttgctcgtcaacatgatgtcgcactacacactacagctcctctctacagtgtgtacatcgaccaaacgacgcatcttttatttctttgtctgttgtgtttgttttcctgtgttcagaacgtacagagccctttaattacctttgctgctcttcacttaacctgataaccagaaggtccagtgctggcgtagccttcacagtttcgtcacgtgtagcgcattccccactttgattggatagggtttttttcctcaagcagattttgtttcacgagttccaccagcaaagcttgtgtcatctgcagctgtaggttggtttgtctaatgagaagaaacaaagctaaacaaaaataaaacacgcaaacaaactcctgaactgattttaaactaattttgatctaaagatcgtaagggaagcaagagcaaacctttttttatgtgtagccccacaccagtttgctggtctttgattaattcagcgagattcagagcacacacacacacacacacacaccg

SEQ ID NO: 34 - protein, 88 aa, Conus textile
MHLSLARSAVLMLLLLFALGNFVVVQSGQITRDVDNGQLTDNRRNLQSKWKPVSLYMSRRGCNNSCQEHSDCESHCICTFRGCGAVNG

(19) United States
(12) Patent Application Publication
(10) Pub. No.: US 2006/0205656 A1
(43) Pub. Date: Sep. 14, 2006

(54) P-superfamily conopeptides

(75) Inventors: David Hooper, Salt Lake City, UT (US); James E. Garrett, Salt Lake City, UT (US); Gloria P. Corpuz, Mililani, HI (US); Lourdes J. Cruz, Manila (PH); Baldomero M. Olivera, Salt Lake City, UT (US); J. Michael McIntosh, Salt Lake City, UT (US); Robert M. Jones, San Diego, CA (US)

Correspondence Address: ROTHWELL, FIGG, ERNST & MANBECK, P.C., 1425 K STREET, N.W., SUITE 800, WASHINGTON, DC, 20005, US

(73) Assignees: Cognetix, Inc., Salt Lake City, UT (US); University of Utah Research Foundation, Salt Lake City, UT (US)

(21) Appl. No.: 11/389,172

(22) Filed: Mar. 27, 2006

Related U.S. Application Data
(63) Continuation of application No. 10/354,047, filed on Jan. 30, 2003, now abandoned.
Application No. 10/354,047 is a division of application No. 09/714,890, filed on Nov. 17, 2000, now abandoned.
(60) Provisional application No. 60/234,762, filed on Sep. 25, 2000.
(60) Provisional application No. 60/165,933, filed on Nov. 17, 1999.

(51) Int. Cl. A61K 38/16; C07K 14/435

(52) U.S. Cl. 514/12; 530/324

(57) ABSTRACT

The present invention is directed to P-superfamily conopeptides, to DNA encoding precursors of the P-superfamily conopeptides and to the precursor peptides.

### CROSS-REFERENCE TO RELATED APPLICATIONS

The present application is a continuation of U.S. patent application Ser. No. 10/354,047 filed 30 Jan. 2003, which in turn is a divisional of U.S. patent application Ser. No. 09/714,890 filed 17 Nov. 2000. U.S. Ser. No.09/714,890 in turn is related to and claims priority under 35 U.S.C. §119(e) to U.S. provisional patent application Ser. No. 60/165,933 filed on 17 Nov. 1999 and to U.S. provisional patent application Ser. No. 60/234,762 filed on 22 Sep. 2000. Each application is incorporated herein by reference.

This invention was made with Government support under Grant No. P01 GM48677 awarded by the National Institutes of Health, Bethesda, Md. The United States Government has certain rights in the invention.

### BACKGROUND OF THE INVENTION

The present invention is directed to P-superfamily conopeptides, to cDNA clones encoding precursors of the P-superfamily conopeptides and to the precursor peptides.

The publications and other materials used herein to illuminate the background of the invention, and in particular, cases to provide additional details respecting the practice, are incorporated by reference, and for convenience are numerically referenced in the following text and respectively grouped in the appended bibliography.

Conus is a genus of predatory marine gastropods (snails) which envenomate their prey. Venomous cone snails use a highly developed apparatus to deliver their cocktail of toxic conotoxins into their prey. In fish-eating species such as Conus magus the cone detects the presence of the fish using chemosensors in its siphon. When close enough extends its proboscis and impales the fish with a hollow harpoon-like tooth containing venom. This immobilizes the fish and enables the cone snail to wind it into its mouth viathe tooth held at the end of its proboscis. For general information on Conus and their venom see the website address http://grimwade.biochem.unimelb.edu.au/cone/referenc.html. Prey capture is accomplished through a sophisticated arsenal of peptides which target specific ion channel and receptor subtypes. Each Conus species venom appears to contain a unique set of 50-200 peptides. The composition of the venom differs greatly between species and between individual snails within each species, each optimally evolved to paralyse it's prey. The active components of the venom are small peptides toxins, typically 10-30 amino acid residues in length and are typically highly constrained peptides due to their high density of disulphide bonds.

The venoms consist of a large number of different peptide components that when separated exhibit a range of biological activities: when injected into mice they elicit a range of physiological responses from shaking to depression. The paralytic components of the venom that have been the focus of recent investigation are the α, ω- and μ-conotoxins. All of these conotoxins act by preventing neuronal communication, but each targets a different aspect of the process to achieve this. The α-conotoxins target nicotinic ligand gated channels, the μ-conotoxins target the voltage-gated sodium channels and the ω-conotoxins target the voltage-gated calcium channels (Olivera et al., 1985; Olivera et al., 1990). For example a linkage has been established between α-, αA-& ψ-conotoxins and the nicotinic ligand-gated ion channel; ω-conotoxins and the voltage-gated calcium channel; μ-conotoxins and the voltage-gated sodium channel; δ-conotoxins and the voltage-gated sodium channel; κ-conotoxins and the voltage-gated potassium channel; conantokins and the ligand-gated glutamate (NMDA) channel.

However, the structure and function of only a small minority of these peptides have been determined to date. For peptides where function has been determined, three classes of targets have been elucidated: voltage-gated ion channels; ligand-gated ion channels, and G-protein-linked receptors.

Conus peptides which target voltage-gated ion channels include those that delay the inactivation of sodium channels, as well as blockers specific for sodium channels, calcium channels and potassium channels. Peptides that target ligand-gated ion channels include antagonists of NMDA and serotonin receptors, as well as competitive and noncompetitive nicotinic receptor antagonists. Peptides which act on G-protein receptors include neurotensin and vasopressin receptor agonists. The unprecedented pharmaceutical selectivity of conotoxins is at least in part defined by specific disulfide bond frameworks combined with hypervariable amino acids within disulfide loops (for a review see McIntosh et al., 1998).

There are drugs used in the treatment of pain, which are known in the literature and to the skilled artisan. See, for example, Merck Manual, 16th Ed. (1992). However, there is a demand for more active analgesic agents with diminished side effects and toxicity and which are non-addictive. The ideal analgesic would reduce the awareness of pain, produce analgesia over a wide range of pain types, act satisfactorily whether given orally or parenterally, produce minimal or no side effects, be free from tendency to produce tolerance and drug dependence.

Due to the high potency and exquisite selectivity of the conopeptides, several are in various stages of clinical development for treatment of human disorders. For example, two Conus peptides are being developed for the treatment of pain. The most advanced is ω-conotoxin MVIIA (ziconotide), an N-type calcium channel blocker (see Heading, C., 1999; U.S. Pat. No. 5,859,186). ω-Conotoxin MVIIA, isolated from Conus magus, is approximately 1000 times more potent than morphine, yet does not produce the tolerance or addictive properties of opiates. ω-Conotoxin MVIIA has completed Phase III (final stages) of human clinical trials and has been approved as a therapeutic agent. ω-Conotoxin MVIIA is introduced into human patients by means of an implantable, programmable pump with a catheter threaded into the intrathecal space. Preclinical testing for use in post-surgical pain is being carried out on another Conus peptide, contulakin-G, isolated from Conus geographus (Craig et al. 1999). Contulakin-G is a 16 amino acid O-linked glycopeptide whose C-terminus resembles neurotensin. It is an agonist of neurotensin receptors, but appears significantly more potent than neurotensin in inhibiting pain in in vivo assays.

In view of a large number of biologically active substances in Conus species it is desirable to further characterize them and to identify peptides capable of treating disorders involving voltage gated ion channels and/or receptors, such as anti-convulsant agents. Surprisingly, and in accordance with this invention, Applicants have discovered novel conotoxins that can be useful for the treatment of disorders involving voltage gated ion channels and/or receptors and could address a long felt need for a safe and effective treatment.

### SUMMARY OF THE INVENTION

In one aspect, the present invention is directed to P-superfamily conopeptides which have the generic sequence

Xaa1-Xaa2-Cys-Xaa3-Xaa4-Xaa5-Xaa6-Cys-Xaa7-Xaa8-Xaa9-Xaa10-Xaa11-Cys-Xaa12-Xaa13-Xaa14-Cys-Xaa15-Xaa16-Cys-Xaa17-Xaa18-Xaa19-Xaa20-Xaa21-Cys-Xaa22-Xaa23-Xaa24-Xaa25-Xaa26-Xaa27-Xaa28 (SEQ ID NO: 1)

where Xaa1 may be Ser, Ala, Asn, Leu, Thr, Gly, g-Thr (g is glycan; i.e., the Thr is glycosylated) or g-Ser; Xaa2 may be des-Xaa2, Ser, Thr, Gly, g-Thr or g-Ser; Xaa3 may be Asn, Gln, Gly, Thr, Ser, g-Thr or g-Ser; Xaa4 may be des-Xaa4 or Gly; Xaa5 may be des-Xaa5, Asn or Asp; Xaa6 may be Ser, Thr, Pro, Hyp (hydroxy-Pro), g-Thr or g-Ser; Xaa7 may be Asn, Gln, Thr, Ser, g-Thr or g-Ser; Xaa8 may be Glu, Ser, Asn, Met, Thr, Gla (γ-carboxy-Glu), Nle (norleucine), Asp, Gln, g-Thr or g-Ser; Xaa9 may be His, Ser, Asp, Thr, g-Thr or g-Ser; Xaa10 may be Ser, Ala, Pro, Hyp, Thr, g-Thr or g-Ser; Xaa11 may be Asp, Glu Gla or any synthetic acidic amino acid; Xaa12 may be des-Xaa12, Glu, Asp, Pro, Hyp, Gla, Ala, Tyr, meta-Tyr, ortho-Tyr, nor-Tyr, mono-halo-Tyr, di-halo-Tyr, O-sulpho-Tyr, O-phospho-Tyr, nitro-Tyr or any synthetic acidic amino acid; Xaa13 may be Ser, Asn, Gly, Thr, Hyp, g-Thr, g-Ser or any synthetic hydroxy containing amino acid; Xaa 14 may be His, Thr, Phe, Asn, lie, Ser, Gin, g-Ser, g-Thr, any synthetic hydroxy containing amino acid, Trp (D or L), neo-Trp, halo-Trp (D or L) or any synthetic aromatic amino acid; Xaa15 may be Ile, Ser, Asp, Glu, Gla, any synthetic amino acid, Thr, g-Ser, g-Thr, any synthetic hydroxy containing amino acid, Tyr, meta-Tyr, ortho-Tyr, nor-Tyr, mono-halo-Tyr, di-halo-Tyr, O-sulpho-Tyr, O-phospho-Tyr or nitro-Tyr; Xaa16 maybe des-Xaa16, Thr, Ser, g-Thr, g-Ser or any synthetic hydroxy containing amino acid; Xaa17 may be des-Xaa17, Asp, Glu, Gla or any synthetic acidic amino acid; Xaa18 may be Thr, Leu, Ile, Val, Ser, g-Thr, g-Ser or any synthetic hydroxy containing amino acid; Xaa 19 may be Phe, His, Gly, Glu, Asp, Gla, any synthetic acidic amino acid, Ser, Thr, g-Ser, g-Thr, any synthetic hydroxy containing amino acid, Trp (D or L), neo-Trp, halo-Trp (D or L) or any synthetic aromatic amino acid; Xaa20 maybe Ser, Thr, Ala, Asp, Asn, Gin, g-Ser, g-Thr, His, Arg, omithine, homo-Lys, homoarginine, nor-Lys, N-methyl-Lys, N,N′-dimethyl-Lys, N,N′,N″-trimethyl-Lys or any synthetic basic amino acid; Xaa21 may be Gly, Gin, Asn, His, Arg, ornithine, homo-Lys, homoarginine, nor-Lys, N-methyl-Lys, N,N′-dimethyl-Lys, N,N′,N″-trimethyl-Lys or any synthetic basic amino acid; Xaa22 may be Gly, Glu, Asp, Gla, any synthetic acidic amino acid, Ile, His, Arg, ornithine, homo-Lys, homoarginine, nor-Lys, N-methyl-Lys, N,N′-dimethyl-Lys, N,N′,N″-trimethyl-Lys or any synthetic basic amino acid; Xaa23 may be des-Xaa23, Ile, Ala, Ser, Pro, Hyp, Phe, Thr, g-Thr, g-Ser or any synthetic hydroxy containing amino acid; Xaa24 may be des-Xaa24, Ile, Val, Thr, Asp, Phe, Ser, g-Thr, g-Ser or any synthetic hydroxy containing amino acid; Xaa25 may be des-Xaa25, Met, Nle, His, Arg, ornithine, homo-Lys, homoarginine, nor-Lys, N-methyl-Lys, N,N′-dimethyl-Lys, N,N′,N″-trimethyl-Lys or any synthetic basic amino acid; Xaa26 may be des-Xaa26, His, Arg, ornithine, homo-Lys, homoarginine, nor-Lys, N-methyl-Lys, N,N′-dimethyl-Lys, N,N′,N″-trimethyl-Lys or any synthetic basic amino acid; Xaa27 may be des-Xaa27, Leu, Asn, Gin, Glu, Asp, Gla or any synthetic amino acid; and Xaa28 may be des-Xaa28, Ile, His, Arg, ornithine, homo-Lys, homoarginine, nor-Lys, N-methyl-Lys, N,N′-dimethyl-Lys, N,N′,N″-trimethyl-Lys or any synthetic basic amino acid. The C-terminus may contain a free carboxyl group or an amide group.

More specifically, the present invention is directed to P-superfamily conopeptides, having the following amino acid sequences:

(SEQ ID NO: 2)

Ser-Cys-Asn-Asn-Ser-Cys-Asn-Xaa1-His-Ser-Asp-Cys-
Xaa1-Ser-His-Cys-Ile-Cys-Thr-Phe-Ser-Gly-Cys-Lys-
Ile-Ile-Leu-Ile;

(SEQ ID NO: 3)

Ser-Cys-Asn-Asn-Ser-Cys-Asn-Xaa1-His-Ser-Asp-Cys-
Xaa1-Ser-His-Cys-Ile-Cys-Thr-Phe-Arg-Gly-Cys-Gly-
Ala-Val-Asn;

(SEQ ID NO: 4)

Ala-Ser-Cys-Gly-Gly-Thr-Cys-Thr-Xaa1-Ser-Ala-Asp-
Cys-Xaa3-Ser-Thr-Cys-Ser-Thr-Cys-Leu-His-Ala-Gln-
Cys-Xaa1-Ser-Thr;

(SEQ ID NO: 5)

Ser-Cys-Gly-Gly-Thr-Cys-Thr-Xaa1-Ser-Ala-Asp-Cys-
Xaa3-Ser-Thr-Cys-Ser-Thr-Cys-Leu-His-Ala-Gln-Cys-
Xaa1;

(SEQ ID NO: 6)

Ala-Cys-Thr-Gly-Ser-Cys-Asn-Ser-Asp-Ser-Xaa1-Cys-
Xaa5-Asn-Phe-Cys-Asp-Cys-Ile-Gly-Thr-Arg-Cys-Xaa1-
Ala-Gln-Lys;

(SEQ ID NO: 7)

Ser-Cys-Asn-Asn-Ser-Cys-Gln-Ser-His-Ser-Asp-Cys-
Ala-Ser-His-Cys-Ile-Cys-Thr-Phe-Arg-Gly-Cys-Gly-
Ala-Val-Asn;

(SEQ ID NO: 8)

Asn-Gly-Cys-Asn-Gly-Asn-Thr-Cys-Ser-Asn-Ser-Xaa3-
Cys-Xaa3-Asn-Asn-Cys-Xaa5-Cys-Asp-Thr-Xaa1-Asp-
Asp-Cys-His-Xaa3-Asp-Arg-Arg-Xaa1-His;

(SEQ ID NO: 9)

Leu-Thr-Cys-Asn-Asp-Xaa3-Cys-Gln-Met-His-Ser-Asp-
Cys-Gly-Ile-Cys-Xaa1-Cys-Val-Xaa1-Asn-Lys-Cys-Ile-
Phe-Phe-Met;

(SEQ ID NO: 10)

Gly-Cys-Asn-Asn-Ser-Cys-Gln-Xaa1-His-Ser-Asp-Cys-
Xaa1-Ser-His-Cys-Ile-Cys-Thr-Phe-Arg-Gly-Cys-Gly-
Ala-Val-Asn;
and

(SEQ ID NO: 11)

Gly-Cys-Asn-Asn-Ser-Cys-Gln-Xaa1-His-Ser-Asp-Cys-
Xaa1-Ser-His-Cys-Ile-Cys-Thr-Ser-Arg-Gly-Cys-Gly-
Ala-Val-Asn,

wherein Xaa1 is Glu or γ-carboxy-Glu; Xaa3 is Pro orhydroxy-Pro; Xaa5 is Tyr, ¹²⁵I-Tyr, mono-iodo-Tyr, di-iodo-Tyr, O-sulpho-Tyr or 0-phospho-Tyr; and the C-terminus contains an amide group or a carboxyl group.

More specifically, the present invention is directed to the following P-Superfamily conopeptides:

Af9.1: SEQ ID NO:2, wherein Xaa1 is Glu;

Af9.2: SEQ ID NO:3, wherein Xaa1 is Glu;

Ca9.1: SEQ ID NO:4, wherein Xaa1 is Glu and Xaa3 is Pro;

Ca9.2: SEQ ID NO:5, wherein Xaa1 is Glu and Xaa3 is Pro;

Cn9.1: SEQ ID NO:6, wherein Xaa1 is Glu and Xaa5 is Tyr;

Gm9.1: SEQ ID NO:7;

Im9 1: SEQ ID NO:8, wherein Xaa1 is Glu, Xaa3 is Pro and Xaa5 is Tyr;

Pn9.1: SEQ ID NO:9, wherein Xaa1 is Glu and Xaa3 is Pro;

tx9a (Tx9.1): SEQ ID NO:10, wherein Xaa1 is Glu or GIa; and

U030: SEQ ID NO: I 1, wherein Xaa1 is Glu or Gla.

The C-terminus of Af9.1, Ca9.1, Ca9.2, Cn9.1, Im9.1 and Pn9.1 preferably contains free carboxy group. The C-terminus of Af9.2, Gm9. 1, tx9a and U030 preferably contains an amide group.

The present invention is also directed to derivatives or pharmaceutically acceptable salts of the P-superfamily genus or conopeptides. Examples of derivatives include peptides in which the Arg residues may be substituted by Lys, omithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; and the Asn, Ser, Thr or Hyp residues may be glycosylated. The halogen may be iodo, chloro, fluoro or bromo; preferably iodo for halogen substituted-Tyr and bromo for halogen-substituted Trp. The Tyr residues may also be substituted with the 3-hydroxyl or 2-hydroxyl isomers (meta-Tyr or ortho-Tyr, respectively) and corresponding O-sulpho- and O-phospho-derivatives. The acidic amino acid residues may be substituted with any synthetic acidic amino acid, e.g., tetrazolyl derivatives of Gly and Ala. The aliphatic amino acids may be substituted by synthetic derivatives bearing non-natural aliphatic branched or linear side chains CₙH₂ₙ₊₂ up to and including n=8. The Leu residues may be substituted with Leu (D). The Glu residues may be substituted with Gla. The Gla residues may be substituted with Glu. The Met residues may be substituted with norleucine (Nle). The Cys residues may be in D or L configuration and may optionally be substituted with homocysteine (D or L).

Examples of synthetic aromatic amino acid include, but are not limited to, nitro-Phe, 4-substituted-Phe wherein the substituent is C₁-C₃ alkyl, carboxyl, hyrdroxymethyl, sulphomethyl, halo, phenyl, —CHO, —CN, —SO₃H and -NHAc. Examples of synthetic hydroxy containing amino acid, include, but are not limited to, such as 4-hydroxymethyl-Phe, 4-hydroxyphenyl-Gly, 2,6-dimethyl-Tyr and 5-amino-Tyr. Examples of synthetic basic amino acids include, but are not limited to, N-1-(2-pyrazolinyl)-Arg, 2-(4-piperidinyl)-Gly, 2-(4-piperidinyl)-Ala, 2-[3-(2S)pyrrolidinyl)-Gly and 2-[3-(2S)pyrrolidinyl)-Ala. These and other synthetic basic amino acids, synthetic hydroxy containing amino acids or synthetic aromatic amino acids are described in Building Block Index, Version 3.0 (1999 Catalog, pages 4-47 for hydroxy containing amino acids and aromatic amino acids and pages 66-87 for basic amino acids; see also http://www.amino-acids.com), incorporated herein by reference, by and available from RSP Amino Acid Analogues, Inc., Worcester, Mass. The residues containing protecting groups are deprotected using conventional techniques. Examples of synthetic acid amino acids include those derivatives bearing acidic functionality, including carboxyl, phosphate, sulfonate and synthetic tetrazolyl derivatives such as described by Ornstein et al. (1993) and in U.S. Pat. No. 5,331,001, each incorporated herein by reference.

Optionally, in the conotoxin peptides of the present invention, the Asn residues may be modified to contain an N-glycan and the Ser, Thr and Hyp residues may be modified to contain an O-glycan (e.g., g-N, g-S, g-T and g-Hyp). In accordance with the present invention, a glycan shall mean any N-, S- or O-linked mono-, di-, tri-, poly- or oligosaccharide that can be attached to any hydroxy, amino or thiol group of natural or modified amino acids by synthetic or enzymatic methodologies known in the art. The monosaccharides making up the glycan can include D-allose, D-altrose, D-glucose, D-mannose, D-gulose, D-idose, D-galactose, D-talose, D-galactosamine, D-glucosamine, D-N-acetyl-glucosamine (GlcNAc), D-N-acetyl-galactosamine (GalNAc), D-fucose or D-arabinose. These saccharides may be structurally modified, e.g., with one or more O-sulfate, O-phosphate, O-acetyl or acidic groups, such as sialic acid, including combinations thereof. The gylcan may also include similar polyhydroxy groups, such as D-penicillamine 2,5 and halogenated derivatives thereof or polypropylene glycol derivatives. The glycosidic linkage is beta and 1-4 or 1-3, preferably 1-3. The linkage between the glycan and the amino acid may be alpha or beta, preferably alpha and is 1-.

Core O-glycans have been described by Van de Steen et al. (1998), incorporated herein by reference. Mucin type O-linked oligosaccharides are attached to Ser or Thr (or other hydroxylated residues of the present peptides) by a GalNAc residue. The monosaccharide building blocks and the linkage attached to this first GaINAc residue define the “core glycans,” of which eight have been identified. The type of glycosidic linkage (orientation and connectivities) are defined for each core glycan. Suitable glycans and glycan analogs are described further in U.S. Ser. No. 09/420,797 filed 19 Oct. 1999 and in PCT Application No. PCT/US99/24380 filed 19 Oct. 1999 (PCT Published Application No. WO 00/23092), each incorporated herein by reference. A preferred glycan is Gal(β1→3)GalNAc(α1→).

Optionally, in the conotoxin peptides described above, pairs of Cys residues maybe replaced pairwise with isoteric lactam or ester-thioether replacements, such as Ser/(Glu or Asp), Lys/(Glu or Asp), Cys/(Glu or Asp) or Cys/Ala combinations. Sequential coupling by known methods (Barnay et al., 2000; Hruby et al., 1994; Bitan et al., 1997) allows replacement of native Cys bridges with lactam bridges. Thioether analogs may be readily synthesized using halo-Ala residues commercially available from RSP Amino Acid Analogues.

The present invention is further directed to derivatives of the above peptides and peptide derivatives which are cylic permutations in which the cyclic permutants retain the native bridging pattern of native toxin. See Craik et al. (2001).

In a second aspect, the present invention is further directed to DNA clones encoding the precursors of the biologically-active mature peptides and to the precursor peptides.

In a third aspect, the present invention is further directed to the use of a member of the P-superfamily of conopeptides for screening drugs for anti-convulsant activity. A member of the P-superfamily may also be used to isolate or assay for its receptor.

In a fourth aspect, the present invention is further directed to a method of identifying compounds that mimic the therapeutic activity of P-Superfamily conopeptides, comprising the steps of: (a) conducting a biological assay on a test compound to determine the therapeutic activity; and (b) comparing the results obtained from the biological assay of the test compound to the results obtained from the biological assay of a P-Superfamily conopeptides. The P-Superfamily conopeptide is labeled with any conventional label, preferably a ¹²⁵I radioisotope on an available Tyr. Thus, the invention is also directed to radioiodinated P-Superfamily conopeptides.

### DETAILED DESCRIPTION OF THE PREFERRED EMBODIMENTS

The present invention is directed to P-superfamily conopeptides which have the generic sequence shown above or specific P-superfamily conopeptides disclosed herein or derivatives thereof.

The present invention is further directed to cDNA clones encoding the precursor of the biologically-active mature peptides and to the precursor peptides. The DNA and precursor proteint sequences are set forth in Table 1

The invention is further directed to the use of these peptides for screening drugs for anticonvulsant activity and to isolate and assay receptors.

The isolation and characterization of the conopeptide tx9a (also called spasmodic peptide or tx9.1) is described herein, as well as the isolation of DNA coding for conopeptide tx9a and the isolation of DNA directed to additional members of the P-Superfamily. The corresponding amino acid sequences of the precursor peptides for members of the P-Superfamily, and the mature peptide sequences of are also disclosed. As disclosed herein, tx9a elicits a spastic or spasmotic response when injected into mice. The spastic or spasmotic response is the same as seen in two well known mutant mouse strains, the spastic mouse and the spasmodic mouse. Since tx9a induces spasticity, it is thus known to have high affinity and specificity for a particular receptor and can be used to target this receptor and in assays for this receptor. tx9a and other members of the P-Superfamily are also useful for screening drugs for anticonvulsant activity.

The conopeptides of the present invention are identified by isolation from Conus venom. Alternatively, the conopeptides of the present invention are identified using recombinant DNA techniques by screening cDNA libraries of various Conus species using conventional techniques such as the use of reverse-transcriptase polymerase chain reaction (RT-PCR) or the use of degenerate probes. Primers for RT-PCR are based on conserved sequences in the signal sequence and 3′ untranslated region of the P-superfamily conopeptide genes. Clones which hybridize to these probes are analyzed to identify those which meet minimal size requirements, i.e., clones having approximately 300 nucleotides (for a propeptide), as determined using PCR primers which flank the cDNA cloning sites for the specific cDNA library being examined. These minimal-sized clones are then sequenced. The sequences are then examined for the presence of a peptide having the characteristics noted above for conopeptides. The biological activity of the peptides identified by this method is tested as described herein, in U.S. Pat. No.5,635,347 or conventionally in the art.

These peptides are sufficiently small to be chemically synthesized. General chemical syntheses for preparing the foregoing conopeptides are described hereinafter, along with specific chemical synthesis of conopeptides and indications of biological activities of these synthetic products. Various ones of these conopeptides can also be obtained by isolation and purification from specific Conus species using the techniques described in U.S. Pat. No. 4,447,356 (Olivera et al., 1984), U.S. Pat. No. 5,514,774 (Olivera et al., 1996) and U.S. Pat. No. 5,591,821 (Olivera et al., 1997), the disclosures of which are incorporated herein by reference.

Although the conopeptides of the present invention can be obtained by purification from cone snails, because the amounts of conopeptides obtainable from individual snails are very small, the desired substantially pure conopeptides are best practically obtained in commercially valuable amounts by chemical synthesis using solid-phase strategy. For example, the yield from a single cone snail may be about 10 micrograms or less of conopeptide. By “substantially pure” is meant that the peptide is present in the substantial absence of other biological molecules of the same type; it is preferably present in an amount of at least about 85% purity and preferably at least about 95% purity. Chemical synthesis of biologically active conopeptides depends of course upon correct determination of the amino acid sequence. Thus, the conopeptides of the present invention may be isolated, synthesized and/or substantially pure.

The conopeptides can also be produced by recombinant DNA techniques well known in the art. Such techniques are described by Sambrook et al. (1989). The peptides produced in this manner are isolated, reduced if necessary, and oxidized to form the correct disulfide bonds, if present in the final molecule.

One method of forming disulfide bonds in the conopeptides of the present invention is the air oxidation of the linear peptides for prolonged periods under cold room temperatures or at room temperature. This procedure results in the creation of a substantial amount of the bioactive, disulfide-linked peptides. The oxidized peptides are fractionated using reverse-phase high performance liquid chromatography (HPLC) or the like, to separate peptides having different linked configurations. Thereafter, either by comparing these fractions with the elution of the native material or by using a simple assay, the particular fraction having the correct linkage for maximum biological potency is easily determined. It is also found that the linear peptide, or the oxidized product having more than one fraction, can sometimes be used for in vivo administration because the cross-linking and/or rearrangement which occurs in vivo has been found to create the biologically potent conopeptide molecule. However, because of the dilution resulting from the presence of other fractions of less biopotency, a somewhat higher dosage may be required.

The peptides are synthesized by a suitable method, such as by exclusively solid-phase techniques, by partial solid-phase techniques, by fragment condensation or by classical solution couplings.

In conventional solution phase peptide synthesis, the peptide chain can be prepared by a series of coupling reactions in which constituent amino acids are added to the growing peptide chain in the desired sequence. Use of various coupling reagents, e.g., dicyclohexylcarbodiimide or diisopropyl- carbonyldimidazole, various active esters, e.g., esters of N-hydroxyphthalimide or N-hydroxy-succinimide, and the various cleavage reagents, to carry out reaction in solution, with subsequent isolation and purification of intermediates, is well known classical peptide methodology. Classical solution synthesis is described in detail in the treatise, “Methoden der Organischen Chemie (Houben-Weyl): Synthese von Peptiden,” (1974). Techniques of exclusively solid-phase synthesis are set forth in the textbook, “Solid-Phase Peptide Synthesis,” (Stewart and Young, 1969), and are exemplified by the disclosure of U.S. Pat. No. 4,105,603 (Vale et al., 1978). The fragment condensation method of synthesis is exemplified in U.S. Pat. No. 3,972,859 (1976). Other available syntheses are exemplified by U.S. Pat. No. 3,842,067 (1974) and U.S. Pat. No. 3,862,925 (1975). The synthesis of peptides containing g-carboxyglutamic acid residues is exemplified by Rivier et al. (1987), Nishiuchi et al. (1993) and Zhou et al. (1996). Synthesis of conopeptides have been described in U.S. Pat. No. 4,447,356 (Olivera et al., 1984), U.S. Pat. No. 5,514,774 (Olivera et al., 1996) and U.S. Pat. No. 5,591,821 (Olivera et al., 1997).

Common to such chemical syntheses is the protection of the labile side chain groups of the various amino acid moieties with suitable protecting groups which will prevent a chemical reaction from occurring at that site until the group is ultimately removed. Usually also common is the protection of an α-amino group on an amino acid or a fragment while that entity reacts at the carboxyl group, followed by the selective removal of the α-amino protecting group to allow subsequent reaction to take place at that location. Accordingly, it is common that, as a step in such a synthesis, an intermediate compound is produced which includes each of the amino acid residues located in its desired sequence in the peptide chain with appropriate side-chain protecting groups linked to various ones of the residues having labile side chains.

As far as the selection of a side chain amino protecting group is concerned, generally one is chosen which is not removed during deprotection of the α-amino groups during the synthesis. However, for some amino acids, e.g., His, protection is not generally necessary. In selecting a particular side chain protecting group to be used in the synthesis of the peptides, the following general rules are followed: (a) the protecting group preferably retains its protecting properties and is not split off under coupling conditions, (b) the protecting group should be stable under the reaction conditions selected for removing the α-amino protecting group at each step of the synthesis, and (c) the side chain protecting group must be removable, upon the completion of the synthesis containing the desired amino acid sequence, under reaction conditions that will not undesirably alter the peptide chain.

It should be possible to prepare many, or even all, of these peptides using recombinant DNA technology. However, when peptides are not so prepared, they are preferably prepared using the Merrifield solid-phase synthesis, although other equivalent chemical syntheses known in the art can also be used as previously mentioned. Solid-phase synthesis is commenced from the C-terminus of the peptide by coupling a protected α-amino acid to a suitable resin. Such a starting material can be prepared by attaching an α-amino-protected amino acid by an ester linkage to a chloromethylated resin or a hydroxymethyl resin, or by an amide bond to a benzhydrylamine (BHA) resin or paramethylbenzhydrylamine (MBHA) resin. Preparation of the hydroxymethyl resin is described by Bodansky et al. (1966). Chloromethylated resins are commercially available from Bio Rad Laboratories (Richmond, Calif.) and from Lab. Systems, Inc. The preparation of such a resin is described by Stewart and Young (1969). BHA and MBHA resin supports are commercially available, and are generally used when the desired polypeptide being synthesized has an unsubstituted amide at the C-terminus. Thus, solid resin supports may be any of those known in the art, such as one having the formulae —O—CH₂-resin support, —NH BHA resin support, or —NH-MBHA resin support. When the unsubstituted amide is desired, use of a BHA or MBHA resin is preferred, because cleavage directly gives the amide. In case the N-methyl amide is desired, it can be generated from an N-methyl BHA resin. Should other substituted amides be desired, the teaching of U.S. Pat. No.4,569,967 (Kornreich et al., 1986) can be used, or should still other groups than the free acid be desired at the C-terminus, it may be preferable to synthesize the peptide using classical methods as set forth in the Houben-Weyl text (1974).

The C-terminal amino acid, protected by Boc or Fmoc and by a side-chain protecting group, if appropriate, can be first coupled to a chloromethylated resin according to the procedure set forth in Horiki et al. (1978), using KF in DMF at about 60° C. for 24 hours with stirring, when a peptide having free acid at the C-terminus is to be synthesized. Following the coupling of the tert-Boc-protected amino acid to the resin support, the α-amino protecting group is removed, as by using trifluoroacetic acid (TFA) in methylene chloride or TFA alone. The deprotection is carried out at a temperature between about 0° C. and room temperature. Other standard cleaving reagents, such as HCl in dioxane, and conditions for removal of specific α-amino protecting groups may be used as described in Schroder and Lubke (1965).

After removal of the α-amino-protecting group, the remaining α-amino- and side chain-protected amino acids are coupled step-wise in the desired order to obtain the intermediate compound defined hereinbefore, or as an alternative to adding each amino acid separately in the synthesis, some of them may be coupled to one another prior to addition to the solid phase reactor. Selection of an appropriate coupling reagent is within the skill of the art. Particularly suitable as a coupling reagent is N,N′-dicyclohexylcarbodiimide (DCC, DIC, HBTU, HATU, TBTU in the presence of HoBt or HoAt).

The activating reagents used in the solid phase synthesis of the peptides are well known in the peptide art. Examples of suitable activating reagents are carbodiimides, such as N,N′-diisopropylcarbodiimide and N-ethyl-N′-(3-dimethylaminopropyl)carbodiimide. Other activating reagents and their use in peptide coupling are described by Schroder and Lubke (1965) and Kapoor (1970).

Each protected amino acid or amino acid sequence is introduced into the solid-phase reactor in about a twofold or more excess, and the coupling may be carried out in a medium of dimethylformamide (DMF):CH₂Cl₂ (1:1) or in DMF or CH₂Cl₂ alone. In cases where intermediate coupling occurs, the coupling procedure is repeated before removal of the α-amino protecting group prior to the coupling of the next amino acid. The success of the coupling reaction at each stage of the synthesis, if performed manually, is preferably monitored by the ninhydrin reaction, as described by Kaiser et al. (1970). Coupling reactions can be performed automatically, as on a Beckman 990 automatic synthesizer, using a program such as that reported in Rivier et al. (1978).

After the desired amino acid sequence has been completed, the intermediate peptide can be removed from the resin support by treatment with a reagent, such as liquid hydrogen fluoride or TFA (ifusing Fmoc chemistry), which not only cleaves the peptide from the resin but also cleaves all remaining side chain protecting groups and also the α-amino protecting group at the N-terminus if it was not previously removed to obtain the peptide in the form of the free acid. If Met is present in the sequence, the Boc protecting group is preferably first removed using trifluoroacetic acid (TFA)/ethanedithiol prior to cleaving the peptide from the resin with HF to eliminate potential S-alkylation. When using hydrogen fluoride or TFA for cleaving, one or more scavengers such as anisole, cresol, dimethyl sulfide and methylethyl sulfide are included in the reaction vessel.

Cyclization of the linear peptide is preferably affected, as opposed to cyclizing the peptide while a part of the peptido-resin, to create bonds between Cys residues. To effect such a disulfide cyclizing linkage, fully protected peptide can be cleaved from a hydroxymethylated resin or a chloromethylated resin support by ammonolysis, as is well known in the art, to yield the fully protected amide intermediate, which is thereafter suitably cyclized and deprotected. Alternatively, deprotection, as well as cleavage of the peptide from the above resins or a benzhydrylamine (BHA) resin or a methylbenzhydrylamine (MBHA), can take place at 0° C. with hydrofluoric acid (HF) or TFA, followed by oxidation as described above. A suitable method for cyclization is the method described by Cartier et al. (1996).

Muteins, analogs or active fragments, of the foregoing t-conotoxin peptides are also contemplated here. See, e.g., Hammerland et al (1992). Derivative muteins, analogs or active fragments of the conotoxin peptides may be synthesized according to known techniques, including conservative amino acid substitutions, such as outlined in U.S. Pat. No. 5,545,723 (see particularly col. 2, line 50 to col. 3, line 8); U.S. Pat. No. 5,534,615 (see particularly col. 19, line 45 to col. 33); and U.S. Pat. No. 5,364,769 (see particularly col. 4, line 55 to col. 7, line 26), each incorporated herein by reference.

Pharmaceutical compositions containing a compound of the present invention as the active ingredient can be prepared according to conventional pharmaceutical compounding techniques. See, for example, Remington's Pharmaceutical Sciences, 18th Ed. (1990, Mack Publishing Co., Easton, Pa.). Typically, an antagonistic amount of active ingredient will be admixed with a pharmaceutically acceptable carrier. The carrier may take a wide variety of forms depending on the form of preparation desired for administration, e.g., intravenous, oral, parenteral or intrathecally. For examples of delivery methods see U.S. Pat. No. 5,844,077, incorporated herein by reference.

“Pharmaceutical composition” means physically discrete coherent portions suitable for medical administration. “Pharmaceutical composition in dosage unit form” means physically discrete coherent units suitable for medical administration, each containing a daily dose or a multiple (up to four times) or a sub-multiple (down to a fortieth) of a daily dose of the active compound in association with a carrier and/or enclosed within an envelope. Whether the composition contains a daily dose, or for example, a half, a third or a quarter of a daily dose, will depend on whether the pharmaceutical composition is to be administered once or, for example, twice, three times or four times a day, respectively.

The term “salt”, as used herein, denotes acidic and/or basic salts, formed with inorganic or organic acids and/or bases, preferably basic salts. While pharmaceutically acceptable salts are preferred, particularly when employing the compounds of the invention as medicaments, other salts find utility, for example, in processing these compounds, or where non-medicament-type uses are contemplated. Salts of these compounds may be prepared by art-recognized techniques.

Examples of such pharmaceutically acceptable salts include, but are not limited to, inorganic and organic addition salts, such as hydrochloride, sulphates, nitrates or phosphates and acetates, trifluoroacetates, propionates, succinates, benzoates, citrates, tartrates, fumarates, maleates, methane-sulfonates, isothionates, theophylline acetates, salicylates, respectively, or the like. Lower alkyl quaternary ammonium salts and the like are suitable, as well.

As used herein, the term “pharmaceutically acceptable” carrier means a non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Wetting agents, emulsifiers and lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. Examples of pharmaceutically acceptable antioxidants include, but are not limited to, water soluble antioxidants such as ascorbic acid, cysteine hydrochloride, sodium bisulfite, sodium metabisulfite, sodium sulfite, and the like; oil soluble antioxidants, such as ascorbyl palmitate, butylated hydroxyanisole (BHA), butylated hydroxytoluene (BHT), lecithin, propyl gallate, aloha-tocopherol and the like; and the metal chelating agents such as citric acid, ethylenediamine tetraacetic acid (EDTA), sorbitol, tartaric acid, phosphoric acid and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid.

A variety of administration routes are available. The particular mode selected will depend of course, upon the particular drug selected, the severity of the disease state being treated and the dosage required for therapeutic efficacy. The methods of this invention, generally speaking, may be practiced using any mode of administration that is medically acceptable, meaning any mode that produces effective levels of the active compounds without causing clinically unacceptable adverse effects. Such modes of administration include oral, rectal, sublingual, topical, nasal, transdermal or parenteral routes. The term “parenteral” includes subcutaneous, intravenous, epidural, irrigation, intramuscular, release pumps, or infusion.

For example, administration of the active agent according to this invention may be achieved using any suitable delivery means, including:

(a) pump (see, e.g., Luer & Hatton (1993), Zimm et al. (1984) and Ettinger et al. (1978));

(b), microencapsulation (see, e.g., U.S. Pat. Nos. 4,352,883; 4,353,888; and 5,084,350);

(c) continuous release polymer implants (see, e.g., U.S. Pat. No. 4,883,666);

(d) macroencapsulation (see, e.g., U.S. Pat. Nos. 5,284,761, 5,158,881, 4,976,859 and 4,968,733 and published PCT patent applications WO92/19195, WO 95/05452);

(e) naked or unencapsulated cell grafts to the CNS (see, e.g., U.S. Pat. Nos. 5,082,670 and 5,618,531);

(f) injection, either subcutaneously, intravenously, intra-arterially, intramuscularly, or to other suitable site; or

(g) oral administration, in capsule, liquid, tablet, pill, or prolonged release formulation.

In one embodiment of this invention, an active agent is delivered directly into the CNS, preferably to the brain ventricles, brain parenchyma, the intrathecal space or other suitable CNS location, most preferably intrathecally.

Alternatively, targeting therapies may be used to deliver the active agent more specifically to certain types of cell, by the use of targeting systems such as antibodies or cell specific ligands. Targeting may be desirable for a variety of reasons, e.g. if the agent is unacceptably toxic, or if it would otherwise require too high a dosage, or if it would not otherwise be able to enter the target cells.

The active agents, which are peptides, can also be administered in a cell based delivery system in which a DNA sequence encoding an active agent is introduced into cells designed for implantation in the body of the patient, especially in the spinal cord region. Suitable delivery systems are described in U.S. Patent No. 5,550,050 and published PCT Application Nos. WO 92/19195, WO 94/25503, WO 95/01203, WO 95/05452, WO 96/02286, WO 96/02646, WO 96/40871, WO 96/40959 and WO 97/12635. Suitable DNA sequences can be prepared synthetically for each active agent on the basis of the developed sequences and the known genetic code.

The active agent is preferably administered in an therapeutically effective amount. By a “therapeutically effective amount” or simply “effective amount” of an active compound is meant a sufficient amount of the compound to treat the desired condition at a reasonable benefit/risk ratio applicable to any medical treatment. The actual amount administered, and the rate and time-course of administration, will depend on the nature and severity of the condition being treated. Prescription of treatment, e.g. decisions on dosage, timing, etc., is within the responsibility of general practitioners or spealists, and typically takes account of the disorder to be treated, the condition of the individual patient, the site of delivery, the method of administration and other factors known to practitioners. Examples o f techniques and protocols can be found in Remington's Pharmaceutical Sciences.

Dosage may be adjusted appropriately to achieve desired drug levels, locally or systemically. Typically the active agents of the present invention exhibit their effect at a dosage range from about 0.001 mg/kg to about 250 mg/kg, preferably from about 0.01 mg/kg to about 100 mg/kg of the active ingredient, more preferably from a bout 0.05 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved. In the event that the response in a subject is insufficient at such doses, even higher doses (or effective higher doses by a different, more localized delivery route) may be employed to the extent that patient tolerance permits. Continuous dosing over, for example 24 hours or multiple doses per day are contemplated to achieve appropriate systemic levels of compounds.

Advantageously, the compositions are formulated as dosage units, each unit being adapted to supply a fixed dose of active ingredients. Tablets, coated tablets, capsules, ampoules and suppositories are examples of dosage forms according to the invention.

It is only necessary that the active ingredient constitute an effective amount, i.e., such that a suitable effective dosage will be consistent with the dosage form employed in single or multiple unit doses. The exact individual dosages, as well as daily dosages, are determined according to standard medical principles under the direction of a physician or veterinarian for use humans or animals.

The pharmaceutical compositions will generally contain from about 0.0001 to 99 wt. %, preferably about 0.001 to 50 wt. %, more preferably about 0.01 to 10 wt. % of the active ingredient by weight of the total composition. In addition to the active agent, the pharmaceutical compositions and medicaments can also contain other pharmaceutically active compounds. Examples of other pharmaceutically active compounds include, but are not limited to, analgesic agents, cytokines and therapeutic agents in all of the major areas of clinical medicine. When used with other pharmaceutically active compounds, the conopeptides of the present invention may be delivered in the form of drug cocktails. A cocktail is a mixture of any one of the compounds useful with this invention with another drug or agent. In this embodiment, a common administration vehicle (e.g., pill, tablet, implant, pump, injectable solution, etc.) would contain both the instant composition in combination supplementary potentiating agent. The individual drugs of the cocktail are each administered in therapeutically effective amounts. A therapeutically effective amount will be determined by the parameters described above; but, in any event, is that amount which establishes a level of the drugs in the area of body where the drugs are required for a period of time which is effective in attaining the desired effects.

Since the P-Superfamily conopeptides cause spastic or spasmotic responses in mice, these peptides are useful for screening drugs for anti-convulsant activity. In accordance with one embodiment of this aspect of the invention, a drug candidate and a P-Superfamily conopeptide are administered to a mouse and the response is monitored. If the drug candidate prevents a spastic or spasmotic response normally seen with the administration of the P-Superfamily conopeptide, then the drug has anticonvulsant activity and can be used to treat convulsions, including epilepsy.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982; Sambrook et al., 1989; Ausubel et al., 1992; Glover, 1985; Anand, 1992; Guthrie and Fink, 1991; Harlow and Lane, 1988; Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986).

### EXAMPLES

The present invention is further detailed in the following Examples, which are offered by way of illustration and are not intended to limit the invention in any manner. Standard techniques well known in the art or the techniques specifically described below are utilized.

### Example 1

### Methods

Purification of the Spasmodic Peptide. The spasmodic peptide was purified from C. textile venom by two different methods:

Purification L Freeze-dried C. textile venom was extracted with 0.2 M ammonium acetate and then fractionated in a Bio-Gel column as described by Hillyard et al. (1989). The spasmodic peptide and the previously described King Kong peptide (δ-conotoxin TxVIA) came from the same size fraction. An early peak was chromatographed on a Vydac reverse-phase C₁₈ column using a gradient of acetonitrile (ACN) in 0.1% trifluoracetic acid (TFA). The resulting major peak was rerun on the same column-buffer system to obtain the pure peptide, which was reduced and alkylated and used for amino acid sequence analysis.

Purification II. Lyophilized C. textile venom from specimens collected in the Philippines (125 mg) was extracted sequentially with 10 mL each of H2O, 20% ACN, 40% ACN, 60% ACN, and 90% ACN. The mixture was sonicated for three 30-s periods over ice water and centrifuged at 5000 g for 5 min; the supernatants were stored at −20° C. The crude venom extract was applied to a preparative scale reversed-phase HPLC; the extract (20 mL) was diluted to 350 mL with 0.1% TFA solution and applied to a C₁₈ Vydac preparative column (22.0×250 mm). Fractions were eluted at 20 mL/min with a linear gradient of 0.1% TFA in water and 0.09% TFA in 60% acetonitrile. Further purification of the peptide used C₁₈ Microsorb MV and C₁₈ Vydac analytical columns at a gradient of 0.23% acetonitrile/min and a flow rate of 1 mL/min. The effluents were monitored at 220 nm, fractions were collected in polypropylene tubes, and aliquots were assayed for biological activity.

Reduction and Alkylation. The purified peptide (1.2 nmol) was reduced with dithiothreitol (DTT) and alkylated with 4-vinylpyri dine. The pH of the peptide solution was adjusted to 8 with 0.5 M tris(hydroxymethyl)amino methane, and DTT was added to a final concentration of 10 mM. The solution was flushed with nitrogen, incubated at 65° C. for 15 min, and cooled to room temperature. Five microliters of 4-vinylpyridine was added per milliliter of solution; the mixture was left in the dark at room temperature for 25 min and then diluted with 500 μL of 0.1% TFA. The mixture was applied on an analytical C₁₈ Microsorb MV HPLC column, which was eluted using 0.1% TFA and 0.085% TFA in 90% acetonitrile (B90) as limiting buffers. The alkylated peptide was recovered by first eluting the column for 50 min with 12% buffer B90 to remove most of the reaction byproducts before applying a gradient of 12-90% buffer B90 over 78 min at a flow rate of 1 mL/min. A blank reaction (without peptide) was run on HPLC for comparison.

Sequencing. The alkylated peptide (300 pmol) was sequenced by standard Edman methods using Applied Bio-system model 492 sequenator (DNA/Peptide Facility, University of Utah). The 3-phenyl-2-thiohydantoin derivatives were identified by HPLC. The sequence was confirmed by mass spectrometry.

Cloning the Spasmodic Peptide. On the basis of the amino acid sequence of the isolated spasmodic peptide from C. textile, oligonucleotide primers were designed for PCR amplification of the corresponding cDNA from a directionally cloned cDNA library (Woodward et al., 1990). Three oligonucleotide primers with degenerate nucleotide sequences were synthesized.

| | |
|---|---|
| Primer 1, | |
| 5′ CCR TTI ACI GCI CCR CAI CC 3′; | (SEQ ID NO: 12) |
| primer 2, | |
| 5′ TGR CAI SWR TTR TTR CAI CC 3′; | (SEQ ID NO: 13) |
| and | |
| primer 3, | |
| ATR CAR TGI SWY TCR CAR TC 3′ | (SEQ ID NO: 14) |

(where 1=inosine, R=A and G, Y=C and T, S=G and C, and W=A and T) represent sequences complementary to the coding sequences at the C-terminus, central, and N-terminus of the peptide, respectively. Primary amplification was carried out using a vector-specific 5′ oligonucleotide and primer 1 in a 1605 Air Thermo-Cycler (Idaho Technology, Idaho Falls, Id.). The product was reamplified using the 5′ vector-specific primer and primer 2 and then electrophoresed on an agarose gel. The major product isolated using Qiaquick gel extraction kit (Qiagen, Valencia, Calif.) was ligated to pGEM-T vector DNA (Promega, Madison, Wis.) and used to transform Escherichia coli DH5R. The nucleic acid sequence of DNA inserted into pGEM-T was determined at the DNA Sequenc-ing Facility at the University of Utah. An oligonucleotide primer corresponding to 5′ sequences was thus obtained, and a vector-specific 3′ primer was used to PCR amplify the entire clone. The amplified DNA was cloned and sequenced. The entire sequence of spasmodic cDNA was assembled from the overlapping sequences and is predicted to contain the amino acid sequence of the mature spasmodic toxin.

cDNA corresponding to the spasmodic peptide from C. gloriamaris was obtained by PCR amplification of DNA isolated from a directionally cloned C. gloriamaris cDNA library. Oligonucleotide primers corresponding to the 5′ and the 3′ untranslated regions of the previously isolated C. textile spasmodic peptide cDNA were used. The amplified DNA was cloned and its sequence determined as described above.

γ-Glutamyl Carboxylase Assay. The peptide corresponding to the −20 to −1 region of the spasmodic peptide precursor, linked at its C-terminus to the pentapeptide FLEEL-NH₂ (SEQ ID NO: 15), was synthesized by Dr. R. Schackmann, DNA Peptide Facility, Huntsman Cancer Center, University of Utah. The identity of the peptide was confirmed using ESI-MS. Partially purified γ-carboxyglutamate carboxylase was prepared as described by Stanley et al. (1997). The assay was carried out as described by Bandyopadhyay et al. (1998), except that the spasmodic peptide pro region (−20 to −1)·FLEEL-NH2 was used as the substrate for the reaction. Experiments were done in triplicate, and the data were fit to a single-site binding model and analyzed using GraphPad Prism from GraphPad Software, Inc. (San Diego, Calif.).

Bioassay. The biological activity of the peptide was determined using 9-15-day-old mice. Approximately 5-290 pmol (per gram body weight) of the lyophilized samples dissolved in normal saline solution were injected i.c. (intracerebral) into mice. Control mice were injected with equal volume of normal saline solution containing dissolved residue (if any) of the corresponding lyophilized column buffer. After injection, the mice were returned to their cages and observed for the onset of any abnormal behavior.

Siamese fighting fish were injected in the dorsal muscle with 10 μL of the saline solution of the peptide and observed for suppression of aggressive behavior when placed in mirrored aquaria. Likewise, control fish were injected with normal saline solution using 26-gauge insulin syringes. Each fish was observed for I h or longer depending on the activity.

### Example 2

### Purification of the Spasmodic Peptide.

The spasmodic peptide was initially detected as an early-eluting major peak from crude Conus textile venom, which was notable for a characteristic suite of symptoms observed after i.c. injection into mice. Within a certain dose range, injected mice were hypersensitive to sensory input and, when either touched or exposed to auditory stimulation, became hyperexcitable to the point where seizure-like symptoms could be induced. Since this symptomatology is characteristic of mutant mice strains carrying either the spasmodic or spastic mutation, we trivially refer to this peptide as the “spasmodic peptide”. The peptide was identified through the various purification steps by following the spasmodic symptomatology described above.

When the purified peptide was injected into mice, even a dose of ≈10 pmol/g was sufficient to induce running in circles and hyperactivity. At higher doses (50 pmol/g), the mice exhibited running and climbing symptoms for close to 1 h. Between 130 and 150 pmol/g, characteristic “spasmodic” symptomatology was elicited. A hand clap would make mice jump high and start running rapidly. When exposed to a loud hand clap, or if the cage cover were dropped, the mice lost motor control and exhibited seizure-like symptoms from which they eventually recovered. At the highest doses tested (>250 pmol/g body weight), after the characteristic spas-modic symptomatology, lethality occurred. Injection of a similar dose range intramuscularly into fish elicited no unusual symptomatology.

### Example 3

### Biochemical Characterization of the Spasmodic Peptide; cDNA Cloning

The amino acid sequence of two batches of purified peptide was determined using standard Edman chemistry. Purified peptide was reduced and alkylated, and a single unequivocal sequence could be assigned through 27 Edman steps, except that no assignment could be made for positions 8 and 13: GCNNSCQXHSDCXSHCICTFRGCGAVN (SEQ ID NO:16, where X meant no assignment could be made). However, a trace of Glu was detected at the two unassigned positions, characteristic of residues that have been posttranslationally modified from glutamate to γ-carboxyglutamate. The presence of γ-carboxyglutamate in the peptide was directly confirmed by alkaline hydrolysis as previously described (McIntosh et al., 1984).

To definitively establish the sequence of the spasmodic peptide, a cDNA clone encoding the spasmodic peptide was identified and characterized from a Conus textile library (Woodward et al., 1990), and a mass spectrometric analysis was carried out. The data in Table 1 show the predicted sequence for the open reading frame from the cDNA clone. This sequence corresponds with amino acid sequence analysis, except for positions 8 and 13 where the cDNA sequence predicts a glutamate residue at both positions, consistent with positions 8 and 13 being γ-carboxyglutamate (Gla) in the mature gene product. The cDNA sequence also predicts that the C-terminal asparagine is amidated (since the C-terminal glycine of the spasmodic peptide precursor would be processed to give an amidated C-terminus in the mature peptide). All of the data taken together are consistent with the following sequence assign-ment for the spasmodic peptide:

Also consistent with the sequence assignment above are the mass spectrometry analyses. Using LDMS, a value of 2955.1 was obtained; an electrospray determination gave a mass of 2955.0. The predicted mass of the mature peptide shown above is 2955.03.

### Example 4

### Evidence for a γ-CRS Sequence in the the Spasmodic Peptide

The presence of γ-carboxyglutamate in the spasmodic peptide suggests that a γ-CRS is docking the γ-carboxylase enzyme at a site N-terminal to the glutamate residues to be posttranslationally modified. It was previously established that the −1 to −20 region of the conantokin-G precursor (another γ-carboxylated conopeptide) contains functional recognition signal sequences (Bandyopadhyay et al., 1998). To test whether the spasmodic peptide precursor from C. textile similarly contains a γ-carboxylation recognition signal sequence in its −1 to −20 region, a peptide chimera was synthesized. The −1 to −20 region from the spasmodic peptide precursor was attached to a model γ-carboxylation substrate FLEEL (SEQ ID NO:15). FLEEL (SEQ ID NO:15), initially designed as a substrate for mammalian γ-glutamyl carboxylase (Suttie et al., 1979), has previously been used for the study of Conus carboxylase (Stanley et al., 1997; Bandyopadhyay et al., 1998; Haushka et al., 1988; Czerwiec et al., 1996). The γ-carboxylation of FLEEL (SEQ ID NO: 15) could then be assessed in the absence and presence of the −1 to −20 region of the spasmodic peptide. Clearly, the presence of the −1 to −20 spasmodic peptide region does indeed increase the affinity for the targeted FLEEL (SEQ ID NO:15) sequence by over 30-fold. The estimated apparent Kmvalues in the absence and presence of propeptide are 1.4×10⁻⁴ and 4.7×10⁻⁶ M, respectively. These results provide evidence for a γ-CRS in the propeptide region of the spasmodic peptide precursor.

### Example 5

### A Conotoxin Related to the Spasmodic Peptide from Conus gloriamaris

In an attempt to characterize other potential members of the spasmodic peptide family, an analysis of other Conus species for cDNA clones related to the spasmodic peptide precursor was carried out. The predicted amino acid sequence of an open reading frame in a cDNA clone from another molluscivorous Conus species, C. gloriamaris, is also shown in Table 1.

The putative sequence of the Conus gloriamaris peptide exhibits a striking level of sequence identity to the spasmodic peptide from C. textile. However, in Conus gloriamaris peptide the two γ-carboxyglutamates of the spasmodic peptide of C. textile are mutated to serine and alanine. Functional differences between the two peptides have not yet been defined, since the peptide from neither C. textile nor C. gloriamaris has been successfully chemically synthesized. However, the results so far indicate that the spasmodic peptide family may be a particularly favorable group to investigate structure/function for peptides containing γ-carboxyglutamate residues.

### Example 6

Isolation of DNA Encoding P-Superfamily Conopeptides

DNA coding for conotoxin peptides was isolated and cloned in accordance with conventional techniques using general procedures well known in the art, such as described in Olivera et al. (1996), including using primers based on the DNA sequence of P-superfamily conopeptides. Alternatively, cDNA libraries were prepared from Conus venom duct using conventional techniques. DNA from single clones was amplified by conventional techniques using primers which correspond approximately to the M13 universal priming site and the M1 3 reverse universal priming site. Clones having a size of approximately 300-500 nucleotides were sequenced and screened for similarity in sequence to known conotoxins. The DNA sequences and encoded propeptide sequences are set forth in Table 1. DNA sequences coding for the mature toxin can also be prepared on the basis of the DNA sequences set forth in Table 1. An alignment of the conopeptides of the present invention is set forth in Table 2.

TABLE 1

| | |
|---|---|
| Name: | Af9.1 |
| Species: | ammiralis |
| Isolated: | No |
| Cloned: | Yes |

| | |
|---|---|
| DNA Sequence: | |
| GTTAAAATGCATCTGTCACTGGCACGCTCAGCTGTTTTGATGTTGCTTCTGCTGTTTGCC | (SEQ ID NO: 17) |
| TTGGGCAACTTTGTTGTGGTCCAGTCAGGACAGATAACAAGAGATGTGGACAATGGAC | |
| AGCTCACGGACAACCGCCGTAACCTGCAATCGAAGTGGAAGCCAGTGAGTCTCTTCAT | |
| GTCACGACGGTCTGTAACAATTCTTGCAATGAGCATTCCGATTGCGAATCCCATTGTA | |
| TTTGCACGTTTAGCGGATGCAAAATTATTTTGATATAAACGGATTGAGTTTGCTCGTCA | |
| ACAAGATGTCGCACTACAGCTCCTCTCTACAGTGTGTACATCGACCAAACGACGCATCT | |
| TTTATTTCTTTGTCTGTTGTATTTGTTTTCCTGTGTTCATAACGTACAGAGCCCTTTAATT | |
| ACCTTTACTGCTCTTCACTTAACCTGATAACCGGAAGGTCCAGTGCT | |
| Translation: | |
| MHLSLARSAVLMLLLLFALGNFVVVQSGQITRDVDNGQLTDNRRNLQSKWKPVSLFMSRR | (SEQ ID NO: 18) |
| SCNNSCNEHSDCESHCICTFSGCKIILI | |
| Toxin Sequence: | |
| Ser-Cys-Asn-Asn-Ser-Cys-Asn-Xaa1-His-Ser-Asp-Cys-Xaa1-Ser-His- | (SEQ ID NO: 2) |
| Cys-Ile-Cys-Thr-Phe-Ser-Gly-Cys-Lys-Ile-Ile-Leu-Ile-\hat{ } | |

| | |
|---|---|
| Name: | Af9.2 |
| Species: | ammiralis |
| Isolated: | No |
| Cloned: | Yes |

| | |
|---|---|
| DNA Sequence: | |
| GTTAAAATGCATCTGTCACTGGCACGCTTAGCTGTTTTGATGTTGCTTCTGCTGTTTGCC | (SEQ ID NO: 19) |
| TTGGGCAACTTTGTTGTGGTCCAGTCAGGACAGATAACAAGAGATGTGGACAATGGAC | |
| AGCTCACGGACAACCGCCGTAACCTGCAATCGAAGTGGAAGCCAGTGAGTCTCTTCAT | |
| GTCACGACGGTCTTGTAACAATTCTTGCAATGAGCATTCCGATTGCGAATCCCATTGTA | |
| TTTGCACGTTTAGAGGATGCGGAGCTGTTAATGGTTGAGTTTGCTCGTCAACATGATGT | |
| CGCACTACACACTACAGCTCCTCTCTACAGTGTGTACATCGACCAAACGACGCATCTTT | |
| TATTTCTTTGTCTGTTGTGTTTGTTTTCCTGTGTTCATAACGTACAGAGCCCTTTAATTAC | |
| TTTTACTGCTCTTCACTTAACCTGATAACCAGAAGGTCCAGTGCT | |
| Translation: | |
| MHLSLARLAVLMLLLLFALGNFVVVQSGQITRDVDNGQLTDNRRNLQSKWKPVSLFMSRR | (SEQ ID NO: 20) |
| SCNNSCNEHSDCESHCICTFRGCGAVNG | |
| Toxin Sequence: | |
| Ser-Cys-Asn-Asn-Ser-Cys-Asn-Xaa1-His-Ser-Asp-Cys-Xaa1-Ser-His- | (SEQ ID NO: 3) |
| Cys-Ile-Cys-Thr-Phe-Arg-Gly-Cys-Gly-Ala-Val-Asn-# | |

| | |
|---|---|
| Name: | Ca9.1 |
| Species: | caracteristicus |
| Isolated: | No |
| Cloned: | Yes |

| | |
|---|---|
| DNA Sequence: | |
| GTTACAATGCATCTGTCACTGGCACGCTCAGCTGTCTTGATGTTGCTTCTGCTGTTTGCC | (SEQ ID NO: 21) |
| TTGGACAACTTCGTTGGGGTCCAGCCAGGACAGATAACAAGAGATGTGGACAACCGCC | |
| GTAACCGGCAATCGCGATGGAAGCCAAGGAGTCTCTTCAAGTCACTTCATAAACGAGC | |
| ATCGTGTGGAGGGACTTGCACGGAAAGTGCCGATTGGCCTTCCACGTGTAGTACTTGCT | |
| TACATGCTCAATGCGAGTCAACATGATGTCGCACTACAGCTCTTCTCTACAGTGTGTAC | |
| ATCGACCGTACGACGCATCTTTTATTTCTTTGGCTGTTTCATTCGTTTTCTTGTGTTCATA | |
| ACATGCGGAGCCCTTCCGTTACCTCTACTGCTCTACACTTAACCTGATAACCAGAAAAT | |
| CCAGTACT | |
| Translation: | |
| MHLSLARSAVLMLLLLFALDNFVGVQPGQITRDVDNRRNRQSRWKPRSLFKSLHKRASCG | (SEQ ID NO: 22) |
| GTCTESADCPSTCSTCLHAQCEST | |
| Toxin Sequence: | |
| Ala-Ser-Cys-Gly-Gly-Thx-Cys-Thr-Xaa1-Ser-Ala-Asp-Cys-Xaa3-Ser- | (SEQ ID NO: 4) |
| Thr-Cys-Ser-Thr-Cys-Leu-His-Ala-Gln-Cys-Xaa1-Ser-Thr-\hat{ } | |

| | |
|---|---|
| Name: | Ca9.2 |
| Species: | caracteristicus |
| Isolated: | No |
| Cloned: | Yes |

| | |
|---|---|
| DNA Sequence: | |
| GTTACAATGCATCTGTCACTGGCACGCTCAGCTGTTTTGATGTTGCTTCTGCTGTTTGCC | (SEQ ID NO: 23) |
| TTGGACAACTTCGTTGGGGTCCAACCAGGACAGATAACTAGAGATGTGGACAACCGCC | |
| GTAACCTGCAATCGCGATGGAAGCCAAGGAGTCTCTTCAAGTCACTTCATAAACGAGC | |
| ATCGTGTGGAGGGACTTGCACGGAAAGTGCCGATTGCCCTTCCACGTGTAGTACTTGCT | |
| TACATGCTCAATGCGAGTGAACATGATGTCGCACTACAGCTCTTCTCTACAGTGTGTAC | |
| ATCGACCGACCGTACGACGCATCTTTTATTTCTTTGTCTGTTTCATTCGTTTTCTTGAGTT | |
| CATAACATGCGGAGCCCTTCCGTTACCTCTACTGCTCTACACTTAAGCTGATAACCAGA | |
| AAATCCAGTACT | |
| Translation: | |
| MHLSLARSAVLMLLLLFALDNFVGVQPGQITRDVDNRRNLQSRWKPRSLFKSLHKRASCG | (SEQ ID NO: 24) |
| GTCTESADCPSTCSTCLHAQCE | |
| Toxin Sequence: | |
| Ser-Cys-Gly-Gly-Thr-Cys-Thr-Xaa1-Ser-Ala-Asp-Cys-Xaa3-Ser-Thr- | (SEQ ID NO: 5) |
| Cys-Ser-Thr-Cys-Leu-His-Ala-Gln-Cys-Xaa1-\hat{ } | |

| | |
|---|---|
| Name: | Cn9.1 |
| Species: | consors |
| Isolated: | No |
| Cloned: | Yes |

| | |
|---|---|
| DNA Sequence: | |
| ATGTTGCTTCTGCTGTTTGCCTTGGGCATCTTCGTTGGGGTCCAGCCAGAACAGATAAC | (SEQ ID NO: 25) |
| AAGAGATGTGGACAAGGGATACTCCACGGATGATGGCCATGACCTGCTATCGCTGTTG | |
| AAGCAAATCAGTCTCCGCGCGTGTACAGGGTCTTGCAATAGTGACAGCGAATGCTACA | |
| ATTTCTGCGACTGCATTGGGACCAGATGTGAGGCACAAAAGTAGACGTCAGAAGAAAA | |
| GGTCCCAGTCGCTCAAGGCAAGAACTAAACGTAGAGAGTTTCCCCGTCAACATGATGT | |
| CGCACTACAACGCTATTCTACTGCGTGTATATCGACCAAACGACGCATCTTTTATTTCTT | |
| TGTCTGTTTGAGTTGTTTTCGTGTGTTCCATTTCCATGACCTTTACTGCCCAACACTTATC | |
| CTGATAACCAGAAGGT | |
| Translation: | |
| MLLLLFALGIFVGVQPEQITRDVDKGYSTDDGHDLLSLLKQISLRACTGSCNSDSECYNFCD | (SEQ ID NO: 26) |
| CIGTRCEAQK | |
| Toxin Sequence: | |
| Ala-Cys-Thr-Gly-Ser-Cys-Asn-Ser-Asp-Ser-Xaa1-Cys-Xaa5-Asn-Phe- | (SEQ ID NO: 6) |
| Cys-Asp-Cys-Ile-Gly-Thr-Arg-Cys-Xaa1-Ala-Gln-Lys-\hat{ } | |

| | |
|---|---|
| Name: | Gm9.1 |
| Species: | gloriamaris |
| Isolated: | No |
| Cloned: | Yes |

| | |
|---|---|
| DNA Sequence: | |
| CCCAGAAAGGAAACACAGCGGTTAAAATGCATCTGTCACTGGCACGCTCAGCTGTTTTG | (SEQ ID NO: 27) |
| ATGTTGCTTCTGCTGTTTGCCTTGGGCAACTTTGTTGTGGTCCAGTCAGGACTGATAACA | |
| AGAGATGTGGACAATGGACAGCTCACGGACAACCGCCGTAACCTGCAAACGGAGTGGA | |
| ACCCATTGAGTCTCTTCATGTCACGACGGTCTTGTAACAATTCTTGCCAGAGCCATTCCG | |
| ATTGCGCATCCCATTGTATTTGCACGTTTAGAGGATGCGGAGCTGTCAATGGTTGAGTT | |
| TGCTCGTCAACATGATGTCGCACTACACACTACAGCTCCTCTCTACAGTGTGTACATCG | |
| ACCAAACGACGCATCTTTTATTTCTTTGTCTGTTGTATTTGTTTTCCTGTGTTCATAACGT | |
| ACAGAGCCCTTTAATTACCTTTACTGCTCTTCAC | |
| Translation: | |
| MHLSLARSAVLMLLLLFALGNFVVVQSGLITRDVDNGQLTDNRRNLQTEWNPLSLFMSRRS | (SEQ ID NO: 28) |
| CNNSCQSHSDCASHCICTFRGCGAVNG | |
| Toxin Sequence: | |
| Ser-Cys-Asn-Asn-Ser-Cys-Gln-Ser-His-Ser-Asp-Cys-Ala-Ser-His-Cys- | (SEQ ID NO: 7) |
| Ile-Cys-Thr-Phe-Arg-Gly-Cys-Gly-Ala-Val-Asn-# | |

| | |
|---|---|
| Name: | Im9.1 |
| Species: | imperialis |
| Isolated: | No |
| Cloned: | Yes |

| | |
|---|---|
| DNA Sequence: | |
| GTTAAAATGCATCTGTCACTGGCAAGCTCAGCTGCTTTGATGTTGCTTCTGCTTTTTGCC | (SEQ ID NO: 29) |
| TTGGGCAACTTCGTTGGGGTCCAGCCAGGACAAATAAGAGATCTGAACAAAGGACAGC | |
| TCAAGGACAACCGCCGTAACCTGCAATCGCAGAGGAAACAAATGAGTCTCCTCAAGTC | |
| ACTTCATGATCGAAATGGGTGTAACGGCAACACGTGTTCCAATAGCCCCTGCCCTAACA | |
| ACTGTTATTGCGATACTGAGGACGACTGCCACCCTGACAGGCGTGAACATTAGAGATTA | |
| GAGAGTTTCCTTGTCAACATGATGTCGCACCACACCTCTGCTCTGCAGTGTGTACATCG | |
| ACCAGTCGACGCATCTGTTATTTCTTTGTCTGTTGGATTGTACATCGACCAGTCCACGCA | |
| TCTGTTATTTCTTTGTCTGTTTGATTTGTTTTCGTGTGTTCATAACACACAGAGCCTTTCT | |
| ATTATCTGTATTGCAATACACTTTGCCTGATAACCAGAAAGTCCAGTGCT | |
| Translation: | |
| MHLSLASSAALMLLLLFALGNFVGVQPGQIRDLNKGQLKDNRRNLQSQRKQMSLLKSLHD | (SEQ ID NO: 30) |
| RNGCNGNTCSNSPCPNNCYCDTEDDCHPDRREH | |
| Toxin Sequence: | |
| Asn-Gly-Cys-Asn-Gly-Asn-Thr-Cys-Ser-Asn-Ser-Xaa3-Cys-Xaa3-Asn- | (SEQ ID NO: 8) |
| Asn-Cys-Xaa5-Cys-Asp-Thr-Xaa1-Asp-Asp-Cys-His-Xaa3-Asp-Arg-Arg- | |
| Xaa1-His-\hat{ } | |

| | |
|---|---|
| Name: | Pn9.1 |
| Species: | pennaceus |
| Isolated: | No |
| Cloned: | Yes |

| | |
|---|---|
| DNA Sequence: | |
| ATGTTGCTTCTGCTGTTTGCCTTGGGCAGCTTCGTTGTGGTCCAGTCAGGACAGATAAC | (SEQ ID NO: 31) |
| AAGAGATGTGGACAATGGGCAGCTCGCGGACAACCGCCGTACCCTGCGATCGCAGTGG | |
| AAGCAAGTGAGTTTCTTCAAGTCACTTGATAAACGACTGACTTGTAACGATCCTTGCCA | |
| GATGCATTCCGATTGCGGCATATGTGAATGCGTGGAAAATAAATGCATATTTTTCATGT | |
| AAACGGATTGAGTTTGCTTGTCAACACAATGTCGCACTGCAGCTCTTCTCTACCGGTGG | |
| GTACATCGACCAAACGACGCATCTTTTATTTCTTTGTCTGTTTCGTTTGTTCTCCTGTGTT | |
| CATAACGTACAGAGCCCTTTAACTACCCTTACTGCTCTTCACTTAACCTGATAACCTGA | |
| AGGTCCGGTGCAGCTGGCGTAGCCTTCACAGTTTCG | |
| Translation: | |
| MLLLLFALGSFVVVQSGQITRDVDNGQLADNRRTLRSQWKQVSFFKSLDKRLTCNDPCQM | (SEQ ID NO: 32) |
| HSDCGICECVENKCIFFM | |
| Toxin Sequence: | |
| Leu-Thr-Cys-Asn-Asp-Xaa3-Cys-Gln-Met-His-Ser-Asp-Cys-Gly-lle- | (SEQ ID NO: 9) |
| Cys-Xaa1-Cys-Val-Xaa1-Asn-Lys-Cys-Ile-Phe-Phe-Met-\hat{ } | |

| | |
|---|---|
| Name: | tx9a (Tx9.1) |
| Species: | textile |
| Isolated: | Yes |
| Cloned: | Yes |

| | |
|---|---|
| DNA Sequence: | |
| ACCCAGAAAGGAAACACAGCGGTTAAAATGCATCTGTCACTGGCACGCTCAGCTGTTTT | (SEQ ID NO: 33) |
| GATGTTGCTTCTGCTGTTTGCCTTGGGCAACTTTGTTGTGGTCCAGTCAGGACAGATAAC | |
| AAGAGATGTGGACAATGGACAGCTCACAGACAACCGCCGTAACCTGCAATCGAAGTGG | |
| AAGCCAGTGAGTCTCTACATGTCACGACGGGGTTGTAACAATTCTTGCCAGGAGCATTC | |
| CGATTGCGAATCCCATTGTATTTGCACGTTTAGAGGATGCGGAGCTGTTAATGGTTGAG | |
| TTTGCTCGTCAACATGATGTCGCACTACACACTACAGCTCCTCTCTACAGTGTGTACATC | |
| GACCAAACGACGCATCTTTTATTTCTTTGTCTGTTGTGTTTGTTTTCCTGTGTTCAGAAC | |
| GTACAGAGCCCTTTAATTACCTTTGCTGCTCTTCACTTAACCTGATAACCAGAAGGTCC | |
| AGTGCTGGCGTAGCCTTCACAGTTTCGTCACGTGTAGCGCATTCCCCACTTTGATTGGAT | |
| AGGGTTTTTTTCCTCAAGCAGATTTTGTTTCACGAGTTCCACCAGCAAAGCTTGTGTCAT | |
| CTGCAGCTGTAGGTTGGTTTGTCTAATGAGAAGAAACAAAGCTAAACAAAAATAAAAC | |
| ACGCAAACAAACTCCTGAACTGATTTTAAACTAATTTTGATCTAAAGATCGTAAGGGAA | |
| GCAAGAGCAAACCTTTTTTTATGTGTAGCCCCACACCAGTTTGCTGGTCTTTGATTAATT | |
| CAGCGAGATTCAGAGCACACACACACACACACACACACCG | |
| Translation: | |
| MHLSLARSAVLMLLLLFALGNFVVVQSGQITRDVDNGQLTDNRRNLQSKWKPVSLYMSRR | (SEQ ID NO: 34) |
| GCNNSCQEHSDCESHCICTFRGCGAVNG | |
| Toxin Sequence: | |
| Gly-Cys-Asn-Asn-Ser-Cys-Gln-Xaa1-His-Ser-Asp-Cys-Xaa1-Ser-His- | (SEQ ID NO: 10) |
| Cys-Ile-Cys-Thr-Phe-Arg-Gly-Cys-Gly-Ala-Val-Asn-# | |

| | |
|---|---|
| Name: | U030 |
| Species: | textile |
| Isolated: | Yes |
| Cloned: | No |

| | |
|---|---|
| DNA Sequence: | |
| Translation: | |
| Toxin Sequence: | |
| Gly-Cys-Asn-Asn-Ser-Cys-Gln-Xaa1-His-Ser-Asp-Cys-Xaa1-Ser-His- | (SEQ ID NO: 11) |
| Cys-Ile-Cys-Thr-Ser-Arg-Gly-Cys-Gly-Ala-Val-Asn-# | |

Xaa1 is Glu or γ-carboxy-Glu
Xaa3 is Pro or hydroxy-Pro
Xaa5 is Tyr, ¹²⁵I-Tyr, mono-iodo-Tyr, di-iodo-Tyr, O-sulpho-Tyr or O-phospho-Tyr
a is free carboxyl or amidated C-terminus, preferably free carboxyl
# is free carboxyl or amidated C-terminus, preferably amidated

TABLE 2
Alignment of P-Superfamily

| | | |
|---|---|---|
| Af9.1 | S-CN-NSCNEHSDCESHCI-C- | (SEQ ID NO: 2) |
| | TFSGCKII--LI\hat{12} | |
| Af9.2 | S-CN-NSCNEHSDCESHCI-C- | (SEQ ID NO: 3) |
| | TFRGCGAV--N# | |
| Ca9.1 | ASCGG-TCTESADCPSTCSTC- | (SEQ ID NO: 4) |
| | LHAQCEST\hat{ } | |
| Ca9.2 | S-CGG-TCTESADCPSTCSTC- | (SEQ ID NO: 5) |
| | LHAQCE\hat{ } | |
| Cn9.1 | A-CTG-SCNSDSECYNFCD-C- | (SEQ ID NO: 6) |
| | IGTRCEA---QK\hat{ } | |
| Im9.1 | NGCNGNTCSNSP-CPNNCY- | (SEQ ID NO: 8) |
| | CDTEDDCHPDRREH\hat{ } | |
| Pn9.1 | LTCN-DPCQMHSDC-GICE-C- | (SEQ ID NO: 9) |
| | VENKCIFFM\hat{ } | |
| U030 | G-CN-NSCQXHSDCXSHCI-C- | (SEQ ID NO: 11) |
| | TSRGCGAV--N# | |
| Tx9.1 | G-CN-NSCQXHSDCXSHCI-C- | (SEQ ID NO: 10) |
| | TFRGCGAV--N# | |
| Gm9.1 | S-CN-NSCQSHSDCASHCI-C- | (SEQ ID NO: 7) |
| | TFRGCGAV--N# | |

X is Glu or Gla.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### LIST OF REFERENCES

Bandyopadhyay, P. K. et al. (1998). J. Biol. Chem. 273:5447-5450.
Bodansky et al. (1966). Chem. Ind. 38:1597-98.
Barnay, G. et al. (2000). J. Med. Chem.
Bitan, G. et al. (1997). J. Peptide Res. 49:421-426.
Cartier, G. E. et al. (1996). J. Biol. Chem. 271:7522-7528.
Craig, A. G. et al. (1999). J. Biol. Chem. 274:13752-13759.
Craik, D. J. et al. (2001). Toxicon 39:43-60.
Czerwiec, E. et al. (1996). Blood 88:2079a.
Ettinger, L. J. et al. (1978). Cancer 41:1270-1273.
Hammerland, L. G. et al. (1992). Eur. J. Pharmacol. 226:239-244.
Hauschka, P. V. et al. (1988). In Current Ad Vances in Vitamin K Research (Suttie, J. W., Ed.) pp 237-243, Science Publishers, New York.
Heading, C. (1999). Curr. Opin. CPNS Invest. Drugs 1:153-166
Hillyard, D. R. et al. (1989). Biochemistry 28:358-361.
Horiki, K. et al. (1978). Chemistry Letters 165-68.
Hubry, V. et al. (1994). Reactive Polymers 22:231-241.
Kaiser et al. (1970). Anal. Biochem. 34:595.
Kapoor(1970). J. Pharm. Sci. 59:1-27.
Kornreich, W. D. et al. (1986). U.S. Pat. No. 4,569,967.
Luer, M. S. & Hatton, J. (1993). Annals Pharmcotherapy 27:912-921.
McIntosh, J. M. et al. (1984). J. Biol. Chem. 259:14343-14346.
McIntosh, J. M. et al. (1998). Methods Enzymol. 294:605-624.
The Merck Manual of Diagnosis and Therapy, 16 Ed., Berkow, R. et al., eds., Merck Research Laboratories, Rahway, N.J., pp. 1436-1445 (1992).
Methoden der Organischen Chemie (Houben-Weyl): Synthese von Peptiden, E. Wunsch (Ed.), Georg Thieme Verlag, Stuttgart, Ger. (1974).
Nishiuchi, Y. et al. (1993). Int. J. Pept. Protein Res. 42:533-538.
Olivera, B. M. et al. (1984). U.S. Pat. No. 4,447,356.
Olivera, B. M. et al. (1985). Science 230:1338-1343.
Olivera, B. M. et al. (1990). Science 249:257-263.
Olivera, B. M. et al. (1996). U.S. Pat. No. 5,514,774.
Olivera, B. M. et al. (1997). U.S. Pat. 5,591,821.
Ornstein, et al. (1993).Biorganic Medicinal Chemistry Letters 3:43-48.
Remington's Pharmaceutical Sciences, 18th Ed., Mack Publishing Co., Easton, Pa. (1990).
Rivier, J. R. et al. (1978). Biopolymers 17:1927-38.
Rivier, J. R. et al. (1987). Biochem. 26:8508-8512.
Sambrook, J. et al. (1979). Molecular Cloning: A Laboratory Manual, 2nd Ed., Cold Spring Harbor Laboratory, Cold Spring Harbor, N.Y.
Shcroder & Lubke (1965). The Peptides 1:72-75, Academic Press, N.Y.
Stanley, T. B. et al. (1997). FEBS Lett. 407:85-88.
Stewart and Young, Solid-Phase Peptide Synthesis, Freeman & Co., San Francisco, Calif. (1969).
Suttie, J. W. et al. (1979). Biochem. Biophys. Res. Commun. 86:500-507.
Vale et al. (1978). U.S. Pat. No. 4,105,603.
Van de Steen, P. et al. (1998). Critical Rev. in Biochem. and Mol. Biol. 33:151-208.
Woodward, S. R. et al. (1 990). EMBO J. 1:1015-1020.
Zimm, S. et al. (1984). Cancer Res. 44:1698-1701.
Zhou L. M., et al. (1996). J. Neurochem. 66:620-628.
U.S. Pat. No. 3,972,859 (1976).
U.S. Pat. No.3,842,067 (1974).
U.S. Pat. 3,862,925 (1975).
U.S. Pat. No. 5,331,001.
U.S. Pat. No. 5,859,186.
PCT Published Application WO 96/11698.
PCT Published Application No. WO 00/23092.

What is claimed is:

1. A substantially pure conopeptide having the sequence
Xaa1-Xaa2-Cys-Xaa3-Xaa4-Xaa5-Xaa6-Cys-Xaa7-Xaa8-Xaa9-Xaa10-Xaa11-Cys-Xaa12-Xaa13-Xaa14-Cys-Xaa15-Xaa16-Cys-Xaa17-Xaa18-Xaa19-Xaa20-Xaa21-Cys-Xaa22-Xaa23-Xaa24-Xaa25-Xaa26-Xaa27-Xaa28
where Xaa1 may be Ser, Ala, Asn, Leu, Thr, Gly, g-Thr or g-Ser; Xaa2 may be des-Xaa2, Ser, Thr, Gly, g-Thr or g-Ser; Xaa3 may be Asn, Gln, Gly, Thr, Ser, g-Thr or g-Ser; Xaa4 may be des-Xaa4 or Gly; Xaa5 may be des-Xaa5, Asn or Asp; Xaa6 may be Ser, Thr, Pro, Hyp (hydroxy-Pro), g-Thr or g-Ser; Xaa7 may be Asn, Gln, Thr, Ser, g-Thr or g-Ser; Xaa8 may be Glu, Ser, Asn, Met, Thr, Gla (γ-carboxy-Glu), Nle (norleucine), Asp, Gln, g-Thr or g-Ser; Xaa9 may be His, Ser, Asp, Thr, g-Thr or g-Ser; Xaa10 may be Ser, Ala, Pro, Hyp, Thr, g-Thr or g-Ser; Xaa11 may be Asp, Glu Gla or any synthetic acidic amino acid; Xaa12 maybe des-Xaa12, Glu, Asp, Pro, Hyp, Gla, Ala, Tyr, meta-Tyr, ortho-Tyr, nor-Tyr, mono-halo-Tyr, ortho-¹²⁵I-Tyr, di-halo-Tyr, O-sulpho-Tyr, O-phospho-Tyr, nitro-Tyr or any synthetic acidic amino acid; Xaa13 may be Ser, Asn, Gly, Thr, Hyp, g-Thr, g-Ser or any synthetic hydroxy containing amino acid; Xaa14 maybe His, Thr, Phe, Asn, Ile, Ser, Gln, g-Ser, g-Thr, any synthetic hydroxy containing amino acid, Trp (D or L), neo-Trp, halo-Trp (D or L) or any synthetic aromatic amino acid; Xaa15 may be Ile, Ser, Asp, Glu, Gla, any synthetic amino acid, Thr, g-Ser, g-Thr, any synthetic hydroxy containing amino acid, Tyr, meta-Tyr, ortho-Tyr, nor-Tyr, ortho-¹²⁵I-Tyr, mono-halo-Tyr, di-halo-Tyr, O-sulpho-Tyr, O-phospho-Tyr or nitro-Tyr; Xaa16 may be des-Xaa16, Thr, Ser, g-Thr, g-Ser or any synthetic hydroxy containing amino acid; Xaa17 may be des-Xaa17, Asp, Glu, Gla or any synthetic acidic amino acid; Xaa18 may be Thr, Leu, Ile, Val, Ser, g-Thr, g-Ser or any synthetic hydroxy containing amino acid; Xaa19 may be Phe, His, Gly, Glu, Asp, Gla, any synthetic acidic amino acid, Ser, Thr, g-Ser, g-Thr, any synthetic hydroxy containing amino acid, Trp (D or L), neo-Trp, halo-Trp (D or L) or any synthetic aromatic amino acid; Xaa20 may be Ser, Thr, Ala, Asp, Asn, Gln, g-Ser, g-Thr, His, Arg, ornithine, homo-Lys, homoarginine, nor-Lys, N-methyl-Lys, N,N′-dimethyl-Lys, N,N′,N″-trimethyl-Lys or any synthetic basic amino acid; Xaa21 may be Gly, Gln, Asn, His, Arg, ornithine, homo-Lys, homoarginine, nor-Lys, N-methyl-Lys, N,N′-dimethyl-Lys, N,N′,N″-trimethyl-Lys or any synthetic basic amino acid; Xaa22 may be Gly, Glu, Asp, Gla, any synthetic acidic amino acid, Ile, His, Arg, ornithine, homo-Lys, homoarginine, nor-Lys, N-methyl-Lys, N,N′-dimethyl-Lys, N,N′,N″-trimethyl-Lys or any synthetic basic amino acid; X23aa maybe des-Xaa23, Ile, Ala, Ser, Pro, Hyp, Phe, Thr, g-Thr, g-Ser or any synthetic hydroxy containing amino acid; Xaa24 may be des-Xaa24, Ile, Val, Thr, Asp, Phe, Ser, g-Thr, g-Ser or any synthetic hydroxy containing amino acid; Xaa25 may be des-Xaa25, Met, Nie, His, Arg, ornithine, homo-Lys, homoarginine, nor-Lys, N-methyl-Lys, N,N′-dimethyl-Lys, N,N′,N″-trimethyl-Lys or any synthetic basic amino acid; Xaa26 may be des-Xaa26, His, Arg, ornithine, homo-Lys, homoarginine, nor-Lys, N-methyl-Lys, N,N′-dimethyl-Lys, N,N′,N″-trimethyl-Lys or any synthetic basic amino acid; Xaa27 may be des-Xaa27, Leu, Asn, Gln, Glu, Asp, Gla or any synthetic amino acid; and Xaa28 may be des-Xaa28, Ile, His, Arg, ornithine, homo-Lys, homoarginine, nor-Lys, N-methyl-Lys, N,N′-dimethyl-Lys, N,N′,N″-trimethyl-Lys or any synthetic basic amino acid.

2. The peptide of claim 1, wherein the six Cys residues from disulfide bridge pairs, whereby the bridged peptide has spasmodic activity.

3. A derivative of the peptide of claim 1, in which the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, omithine, homoargine, nor-Lys, or any synthetic basic amino acid; the Tyr residues may be substituted with meta-Tyr, ortho-Tyr, nor-Tyr, mono-halo-Tyr, di-halo-Tyr, O-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues maybe substituted with Trp (D), neo-Trp, halo-Trp (D or L) (wherby halo is F, Cl, Br, I at the indolic positions 5 or 6 or both) or any aromatic synthetic amino acid; the Asn, Ser, Thr or Hyp residues may be glycosylated;. the Tyr residues may also be substituted with the 3-hydroxyl or 2-hydroxyl isomers (meta-Tyr or ortho-Tyr, respectively) and corresponding O-sulpho- and O-phospho-derivatives; the acidic amino acid residues may be substituted with any synthetic acidic amino acid, e.g., tetrazolyl derivatives of Gly and Ala; the aliphatic amino acids may be substituted by synthetic derivatives bearing non-natural aliphatic branched or linear side chains CₙH₂ₙ₊₂ up to and including n=8; the Leu residues may be substituted with Leu (D); the Glu residues may be substituted with Gla; the Gla residues may be substituted with Glu; the Met residues may be substituted by Nle; the Cys residues may be in D or L configuration and may optionally be substituted with homocysteine (D or L); and pairs of Cys residues may be replaced pairwise with isoteric lactam or ester-thioether replacements, such as Ser/Glu (or Asp), Lys/Glu (or Asp), Cys/Glu (or Asp), Cys/Ala or Cys/Glu (or Asp) combinations.

4. The substantially pure conopeptide of claim 1 selected from the group consisting of:
(SEQ ID NO: 2)
Ser-Cys-Asn-Asn-Ser-Cys-Asn-Xaa1-His-Ser-Asp-Cys-
Xaa1-Ser-His-Cys-Ile-Cys-Thr-Phe-Ser-Gly-Cys-Lys-
Ile-Ile-Leu-Ile;
(SEQ ID NO: 3)
Ser-Cys-Asn-Asn-Ser-Cys-Asn-Xaa1-His-Ser-Asp-Cys-
Xaa1-Ser-His-Cys-Ile-Cys-Thr-Phe-Arg-Gly-Cys-Gly-
Ala-Val-Asn;
(SEQ ID NO: 4)
Ala-Ser-Cys-Gly-Gly-Thr-Cys-Thr-Xaa1-Ser-Ala-Asp-
Cys-Xaa3-Ser-Thr-Cys-Ser-Thr-Cys-Leu-His-Ala-Gln-
Cys-Xaa1-Ser-Thr;
(SEQ ID NO: 5)
Ser-Cys-Gly-Gly-Thr-Cys-Thr-Xaa1-Ser-Ala-Asp-Cys-
Xaa3-Ser-Thr-Cys-Ser-Thr-Cys-Leu-His-Ala-Gln-Cys-
Xaa1;
(SEQ ID NO: 6)
Ala-Cys-Thr-Gly-Ser-Cys-Asn-Ser-Asp-Ser-Xaa1-Cys-
Xaa5-Asn-Phe-Cys-Asp-Cys-IIe-Gly-Thr-Arg-Cys-Xaa1-
Ala-Gln-Lys;
(SEQ ID NO: 7)
Ser-Cys-Asn-Asn-Ser-Cys-Gln-Ser-His-Ser-Asp-Cys-
Ala-Ser-His-Cys-Ile-Cys-Thr-Phe-Arg-Gly-Cys-Gly-
Ala-Val-Asn;
(SEQ ID NO: 8)
Asn-Gly-Cys-Asn-Gly-Asn-Thr-Cys-Ser-Asn-Ser-Xaa3-
Cys-Xaa3-Asn-Asn-Cys-Xaa5-Cys-Asp-Thr-Xaa1-Asp-
Asp-Cys-His-Xaa3-Asp-Arg-Arg-Xaa1-His;
(SEQ ID NO: 9)
Leu-Thr-Cys-Asn-Asp-Xaa3-Cys-Gln-Met-His-Ser-Asp-
Cys-Gly-Ile-Cys-Xaa1-Cys-Val-Xaa1-Asn-Lys-Cys-Ile-
Phe-Phe-Met;
(SEQ ID NO: 10)
Gly-Cys-Asn-Asn-Ser-Cys-Gln-Xaa1-His-Ser-Asp-Cys-
Xaa1-Ser-His-Cys-Ile-Cys-Thr-Phe-Arg-Gly-Cys-Gly-
Ala-Val-Asn;
and
(SEQ ID NO: 11)
Gly-Cys-Asn-Asn-Ser-Cys-Gln-Xaa1-His-Ser-Asp-Cys-
Xaa1-Ser-His-Cys-Ile-Cys-Thr-Ser-Arg-Gly-Cys-Gly-
Ala-Val-Asn,
wherein Xaa1 is Glu or γ-carboxy-Glu; Xaa3 is Pro or hydroxy-Pro; Xaa5 is Tyr, 125I-Tyr, mono-iodo-Tyr, di-iodo-Tyr, O-sulpho-Tyr or O-phospho-Tyr; and the C-terminus contains an amide group or a carboxyl group.

5. The peptide of claim 4, wherein the six Cys residues from disulfide bridge pairs, whereby the bridged peptide has spasmodic activity.

6. A derivative of the peptide of claim 4, in which the Arg residues may be substituted by Lys, ornithine, homoargine, nor-Lys, N-methyl-Lys, N,N-dimethyl-Lys, N,N,N-trimethyl-Lys or any synthetic basic amino acid; the Lys residues may be substituted by Arg, ornithine, homoargine, nor-Lys, or any synthetic basic amino acid; the Tyr residues may be substituted with meta-Tyr, ortho-Tyr, nor-Tyr, mono-halo-Tyr, di-halo-Tyr, 0-sulpho-Tyr, O-phospho-Tyr, nitro-Tyr or any synthetic hydroxy containing amino acid; the Ser residues may be substituted with Thr or any synthetic hydroxylated amino acid; the Thr residues may be substituted with Ser or any synthetic hydroxylated amino acid; the Phe residues may be substituted with any synthetic aromatic amino acid; the Trp residues may be substituted with Trp (D), neo-Trp, halo-Trp (D or L) or any aromatic synthetic amino acid; the Asn, Ser, Thr or Hyp residues may be glycosylated; the Tyr residues may also be substituted with the 3-hydroxyl or 2-hydroxyl isomers (meta-Tyr or ortho-Tyr, respectively) and corresponding O-sulpho- and O-phospho-derivatives; the acidic amino acid residues may be substituted with any synthetic acidic amino acid, e.g., tetrazolyl derivatives of Gly and Ala; the aliphatic amino acids may be substituted by synthetic derivatives bearing non-natural aliphatic branched or linear side chains CnH₂ₙ₊₂ up to and including n=8; the Leu residues may be substituted with Leu (D); the Glu residues may be substituted by Gla; the Gla residues may be substituted by Glu; the Met residues may be substituted by Nle; the Cys residues may be in D or L configuration and may optionally be substituted with homocysteine (D or L); and
pairs of Cys residues may be replaced pairwise with isoteric lactam or ester-thioether replacements, such as Ser/(Glu or Asp), Lys/(Glu or Asp), Cys/(Glu or Asp), Cys/Glu (or Asp) or Cys/Ala combinations.

7. A substantially pure P-Superfamily conopeptide derivative comprising a permutant of the peptide of claim 1.

8. A substantially pure P-Superfamily conopeptide derivative comprising a permutant of the peptide of claim 3.

9. A substantially pure P-Superfamily conopeptide derivative comprising a permutant of the peptide of claim 4.

10. A substantially pure P-Superfamily conopeptide derivative comprising a permutant of the peptide of claim 6.

11. An isolated nucleic acid encoding a P-superfamily conopeptide precursor having an amino acid sequence selected from the groups consisting of the amino acid sequences set forth in SEQ ID NOs:18, 20, 22, 24, 26, 28, 30, 32 and 34.

12. The isolated nucleic acid of claim 11, wherein the nucleic acid comprises a nucleotide sequence selected from the group consisting of the nucleotide sequences set forth in SEQ ID NOs:17, 19,21,23,25, 27,29, 31 and 33.

13. An isolated P-superfamily conopeptide precursor having an amino acid sequence selected from the group consisting of the amino acid sequences set forth in SEQ ID NOs: 18, 20,22, 24, 26, 28, 30, 32 and 34.

14. A method for screening a drug candidate for anti-convulsant activity which comprises (a) administering a P-Superfamily conopeptide of claim 1 and said drug candidate to a mouse and (b) monitoring the response of said mouse, wherein if the drug candidate prevents a spastic or spasmotic response in said mouse, then the drug has anticonvulsant activity.

15. A method for treating convulsions which comprises administering to a patient in need thereof a therapeutically effective amount of a drug identified by the method of claim 14.

16. A method for screening a drug candidate for anti-convulsant activity which comprises (a) administering a P-Superfamily conopeptide of claim 4 and said drug candidate to a mouse and (b) monitoring the response of said mouse, wherein if the drug candidate prevents a spastic or spasmotic response in said mouse, then the drug has anti-convulsant activity.

17. A method for treating convulsions which comprises administering to a patient in need thereof a therapeutically effective amount of a drug identified by the method of claim 26.

18. A method of identifying compounds that mimic the therapeutic activity of a P-Superfamily conopeptide, comprising the steps of: (a) conducting a biological assay on a test compound to determine the therapeutic activity; and (b) comparing the results obtained from the biological assay of the test compound to the results obtained from the biological assay of a P-Superfamily conopeptide of claim 1.

19. A method of identifying compounds that mimic the therapeutic activity of a P-Superfamily conopeptide, comprising the steps of: (a) conducting a biological assay on a test compound to determine the therapeutic activity; and (b) comparing the results obtained from the biological assay of the test compound to the results obtained from the biological assay of a P-Superfamily conopeptide of claim 4.

20. A method for making a pharmaceutical formulation for the treatment of convulsions which comprises:
(a) co-administering candidate compounds and a P-Superfamily conopeptide of claim 1 to a mouse;
(b) selecting a compound identified in step (a) which prevents a spastic or spasmotic response in said mouse;
(c) manufacturing bulk quantities of the compound selected in step (b); and
(d) formulating the compound manufactured in step (c) in a pharmaceutically acceptable carrier.
